(19) United States
(12) Patent Application Publication
(10) Pub. No.: US 2024/0415892 A1
(43) Pub. Date: Dec. 19, 2024

(54) METHOD FOR EXPANSION AND MAINTENANCE OF NK CELLS FOR IMMUNOTHERAPY

(71) Applicant: NKure Therapeutics Private Limited, Bangalore (IN)

(72) Inventors: Rohini Roy, Bangalore (IN); Vandya Juneja Shah, Bangalore (IN); Aditi Ravindra Battu, Bangalore (IN); Renjitha Gopurappilly, Bangalore (IN)

(21) Appl. No.: 18/743,318

(22) Filed: Jun. 14, 2024

(30) Foreign Application Priority Data
Jun. 14, 2023 (IN) 202341040645

(51) Int. Cl. A61K 35/17; A61K 39/00; C12N 5/00; C12N 5/0783; G01N 33/50

(52) U.S. Cl. CPC A61K 35/17; A61K 39/4613; A61K 39/4631; A61K 39/464412; C12N 5/0018; C12N 5/0646; G01N 33/5047; C12N 2501/2302; C12N 2502/1352; C12N 2502/45; C12N 2510/04

(57) ABSTRACT

The present invention discloses a method for expansion of natural killer (NK) cells or CAR-NK cells using peripheral blood mononuclear cells (PBMCs) and culturing the cells in a medium comprising a feeder layer having a population of mesenchymal stem cells (MSCs) with disrupted or deleted MHC class I gene and/or MHC class II gene and a heterologous gene for expressing Interleukin-21 (IL-21), and Interleukin-2 (IL-2). The present disclosure further provides a method and a model for evaluating natural killer (NK) cells or CAR-NK cells using tester line comprising induced Pluripotent stem cells (iPSCs) comprising disrupted or deleted MHC class I gene and/or MHC class II gene and a heterologous gene encoding a tumor antigen.

### CROSS-REFERENCE TO RELATED APPLICATIONS

This application claims the benefit of IN patent application No. 202341040645, filed Jun. 14, 2023, which is herein incorporated by reference in its entirety for all purposes.

### REFERENCE TO A SEQUENCE LISTING SUBMITTED AS AN XML FILE

The Sequence Listing written in file PD050855IN-SC_Sequence Listing.xml is 19,330 bytes, was created on Jun. 12, 2024, and is hereby incorporated by reference.

### FIELD OF INVENTION

The present disclosure relates to the field of natural killer (NK) cells and, particularly to, a method for expansion and maintenance of NK cells. It further relates to a method for production and maintenance of Chimeric antigen receptors expressing natural killer cells (CAR-NK) for immunotherapy. Further, the present disclosure also relates to a method for evaluating CAR-NK cells for immunotherapeutic applications. It further relates to conditionally immortalized feeder cells for expansion of NK cells and CAR-NK cells.

### BACKGROUND OF THE INVENTION

Based on the reports from Globocan 2020, leukemia and non-Hodgkin lymphoma are among the ten most prevalent cancer types in India. Clearly, with more than 50,000 fresh cases being diagnosed, the hematological cancer burden in India is becoming a cause of concern. Given the socio-economic situation of a large population being affected by these diseases, there is a need for an affordable therapeutic solution. In India, Chimeric antigen receptor expressing T cells (CAR-T) cell therapy has been gaining a lot of attention as a prominent therapeutic solution for cancer. However, CAR-T cell therapy is quite expensive to implement because of the costs involved in manufacture and delivery of the final product. To cater the needs of the larger Indian population, an allogenic therapy based on engineered NK cells would be a suitable option.

Engineered natural killer (NK) cells in immunotherapy to specifically target and kill infected cells or tumor cells, without prior sensitization, have received increased attention in recent years. Chimeric antigen receptor (CAR) genes are introduced into NK cells using gene editing tools such as CRISPR/Cas system, transposon gene editing system, etc. for expressing CARs targeting the various surface antigens expressed on tumor or infected cells to induce cell cytotoxicity. The use of NK cells has been considered as a better alternative to T-cell for their accessibility and safety, particularly since NK cell applications exhibit a lower incidence of graft versus host disease (GVHD) (Zhang C, et al. Chimeric antigen receptor-engineered NK-92 cells: An off-the-Shelf cellular therapeutic for targeted elimination of cancer cells and induction of protective antitumor immunity. Front Immunol (2017) 8:533. doi: 10.3389/fimmu.2017.00533; and Shifrin N, Raulet D H, Ardolino M. NK cell self-tolerance, responsiveness and missing self-recognition. Semin Immunol (2014) 26:138 44. doi: 10.1016/j.smim.2014.02.007).

Various strategies have been employed in expansion of allogenic and autologous NK cells, NK cell lines and CAR-NK cells to achieve effective NK cell-based immunotherapies. However, achieving effective expansion of NK cells for commercial manufacture remains a challenge. There is a need for a robust and cost-effective method to achieve preferential expansion of NK cells in large numbers.

### SUMMARY OF INVENTION

In an aspect of the present disclosure, there is provided a method for expansion of natural killer (NK) cells, comprising: (i) obtaining a first population of NK cells; (ii) culturing the cells in a medium to obtain activated NK cells, wherein the medium comprises: (a) a feeder layer having a population of mesenchymal stem cells (MSCs) comprising disrupted or deleted MHC class I gene and/or MHC class II gene and a heterologous gene for expressing Interleukin-21 (IL-21) and (b) Interleukin-2 (IL-2); and (iii) expanding the NK cells by repeatedly replacing the feeder layer and culturing the NK cells, to obtain expanded NK cells.

In an aspect of the present disclosure, there is provided a method for producing CAR-NK cells, comprising: (i) providing NK cells, or the expanded NK cells obtained by the method described hereinabove; (ii) introducing a heterologous gene encoding a chimeric antigen receptor (CAR) gene, to the NK cells to obtain CAR-NK cells; (iii) culturing the CAR-NK cells in a medium to obtain activated CAR-NK cells, wherein the medium comprises: (a) a feeder layer having a population of mesenchymal stem cells (MSCs) comprising disrupted or deleted MHC class I gene and/or MHC class II gene and a heterologous gene for expressing Interleukin-21 (IL-21); and (b) IL-2; and (iv) expanding the activated CAR-NK cells by repeatedly replacing the feeder layer and culturing the CAR-NK cells, to obtain expanded CAR-NK cells.

In an aspect of the present disclosure, there is provided a method for evaluating CAR-NK cells, said method comprising: (a) providing CAR-NK cells or the expanded CAR-NK cells obtained by the method described herein; (b) co-culturing, in-vitro, the expanded CAR-NK cells and a tester cell line in a medium wherein the tester cell line comprises induced Pluripotent stem cells (iPSCs) comprising disrupted or deleted MHC class I gene and/or MHC class II gene and a heterologous gene encoding a tumor antigen; and (c) determining cytotoxicity by measuring cell death or cell damage in the tester cell line.

In an aspect of the present disclosure, there is provided a method for evaluating natural killer (NK) cells, said method comprising: (a) providing an animal comprising an implant of a tester cell line or an animal comprising an implant of tumor, wherein the tester line comprises induced Pluripotent stem cells (iPSCs) comprising disrupted or deleted MHC class I gene and/or MHC class II gene and a heterologous gene encoding a tumor antigen; (b) introducing the NK cells or expanded NK cells obtained by the method described herein, into said animal; and (c) determining cytotoxicity, in vivo, by measuring cell death or cell damage in the tester cell line or the tumor cell mass.

In an aspect of the present disclosure, there is provided a model for evaluating CAR-NK cells, said model comprising a tester line, wherein the tester line comprises induced Pluripotent stem cells (iPSCs) comprising disrupted or deleted MHC class I gene and/or MHC class II gene and a heterologous gene for expressing a tumor antigen.

In an aspect of the present disclosure, there is provided an expanded population of NK or CAR NK cells obtained by a method as disclosed herein, for use in treating a patient in need thereof.

In an aspect of the present disclosure, there is provided a feeder layer for expansion of NK cells or CAR-NK cells, said feeder layer comprising a population of mesenchymal stem cells (MSCs) having disrupted or deleted MHC class I gene and/or MHC class II gene and a heterologous gene for expressing Interleukin-21 (IL-21).

In an aspect of the present disclosure, there is provided a medium for expansion of NK cells or CAR-NK cells, said medium comprising the feeder layer as disclosed herein; and an effective amount of Interleukin-2 (IL-2).

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 is a schematic representation depicting the method for production and expansion of CAR-NK cells, in accordance with the embodiments herein.

FIG. 2 is a schematic representation depicting the method for evaluation of the cytotoxicity of the NK or CAR-NK cells, in accordance with the embodiments herein.

FIG. 3 illustrates the preparation of iPSC having MHC class I (B2M gene) and Class II (CIITA gene) Knockout (KO), in accordance with the embodiments herein.

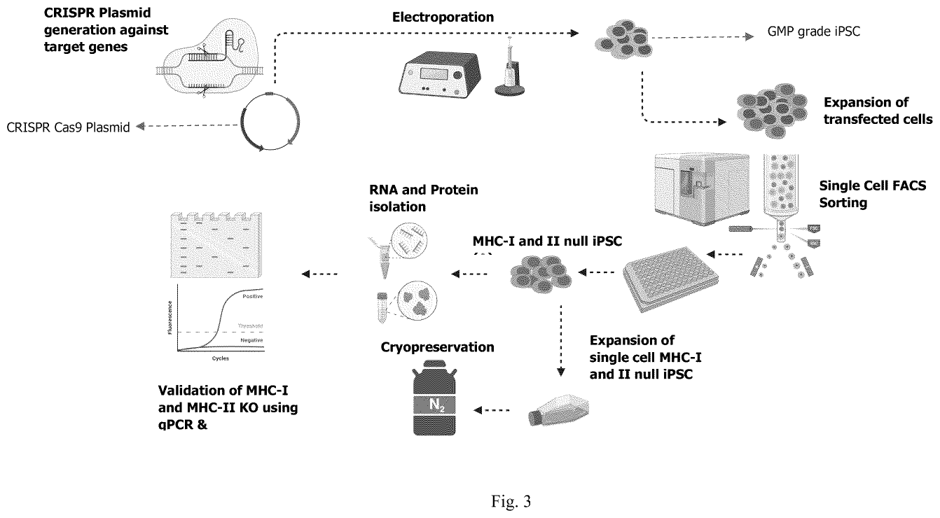

FIG. 4 depicts immortalization of MHC class I and II MSCs, followed by overexpression of membrane bound IL-21, in accordance with the embodiments herein.

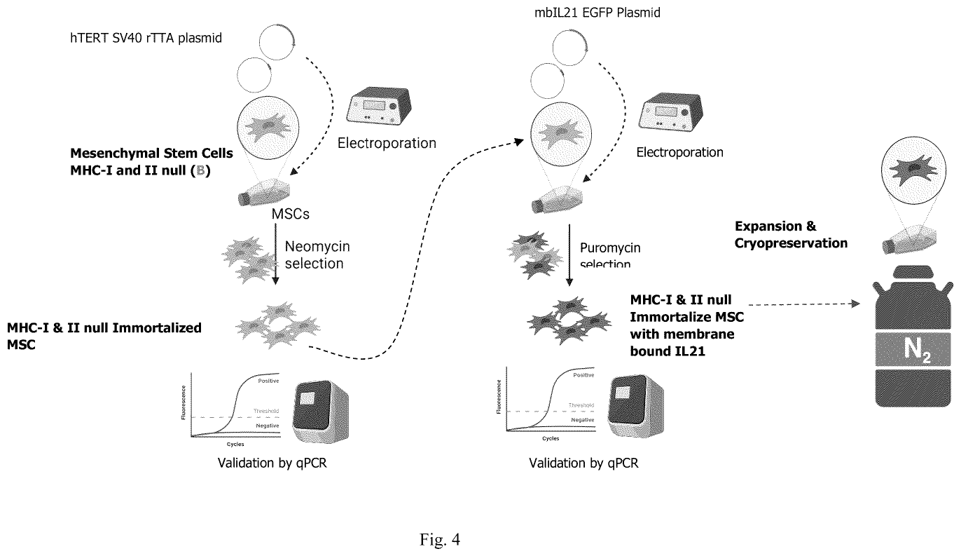

FIG. 5 illustrates MHC-I and II null MSC transfected with hTERT SV40 T-antigen plasmid to achieve immortalization. A. (Top panel) Untransfected control MSCs post neomycin selection at DAY 1 and DAY 5. (Bottom panel) Transfected MSCs post neomycin selection at DAY 1 and DAY 5, followed by expansion of the selected cells. B. qPCR result showing differential expression of hTERT, SV40LT and rtTA compared to control cells, in accordance with the embodiments herein.

FIG. 6, panel A. Representative images showing Mesenchymal Stem cell (MSC) feeder morphology in culture at 24 hours (top) and at 48 hours (bottom) B. Line plot showing the growth pattern of the feeder cells. The population doubling time is ˜40 hours. 2 data points representing 2 experimental repeats. C. Bar graphs showing the expression fold change of membrane bound IL-21 between control and membrane bound IL-21 overexpressing feeder cells at early (P17) and late passages (P36). D. Contour plot showing the flow cytometry results characterizing the MSC cells by expressing CD90 and CD105, in accordance with the embodiments herein.

FIG. 7, panel A, are representative images showing the expansion of PBMC population at Day 1 and Day 14 with feeder; FIG. 7, panel B. Line plots showing fold expansion of NK cells using the feeder layer and method, in different PBMC samples from two donors according to the embodiments herein, at Day 23; FIG. 7, panel C. Contour plot depicting purity of NK cells (Q1) after 14 days of expansion, FIG. 7, panel D. Line plots showing attenuated fold expansion of NK cells without feeders in different PBMC samples from two donors, in accordance with the embodiments herein.

FIG. 8 are images depicting target specific cytotoxicity results of expanded NK cells using the CD19 iPSC tester line, in accordance with the embodiments herein.

FIG. 9, panel A, is a schematic representation depicting luciferase assay system, and FIG. 9, panel B, shows luminescence results, in accordance with the embodiments herein.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of elements or steps but not the exclusion of any other element or step or group of elements or steps.

The term “including” is used to mean “including but not limited to”. “Including” and “including but not limited to” are used interchangeably.

Embodiments herein include a method for expansion and maintenance of immune cells, particularly natural killer (NK) cells. Natural killer cells are increasingly being used in immunotherapeutic applications, and their expansion is crucial for achieving optimal clinical outcome. Accordingly, embodiments herein achieve a method for expansion and maintenance of natural killer cells or engineered natural killer cells for immunotherapy. The method, as disclosed herein, is capable of achieving a multifold expansion of NK cells. Further, the embodiments herein also provide a method for production, expansion, and maintenance of CAR-NK cells. Embodiments herein also provide a tester line for evaluation of NK cells and CAR-NK cells. Accordingly, embodiments herein also include a method for evaluation of NK cells and CAR-NK cells having immunotherapeutic potential.

### Natural Killer Cells

Embodiments herein provide a method for expansion and maintenance of Natural killer cells. The term “natural killer cell” or “NK cell”, as used herein, refers to a lymphocyte of the innate immune system. It is capable of inducing or mediating cytotoxicity in target cells such as tumor cells and infected cells. In general, NK cells are characterized by the presence of CD56 (i.e. CD56⁺) and the absence of CD3 (CD3⁻) surface markers. Accordingly, the NK cells refer to CD 56⁺ and CD3-cells. The NK cells intended for expansion by the method, according to embodiments herein, may be modified cells, for e.g.: including one or more heterologous genes of interest, or unmodified NK cells. The NK cells may be a homogenous or heterogenous population of NK cells. In an embodiment, the NK cells refer to the NK cells present in a mixed population of cells comprising NK cells. Accordingly, preferential expansion or enrichment of NK cells in a mixed population of cells is achieved in various embodiments herein. The NK cells may be obtained from commercial sources or, alternatively, derived from various platforms including Umbilical cord blood (UCB), peripheral blood (PB), white blood, buffy coat, peripheral blood mononuclear cells (PBMCs), induced pluripotent stem cells (iPSCs), and embryonic stem cells. The NK cells, in accordance with the embodiments herein, may be allogenic or autologous, or NK cell line including NK92, NK 3.3, YTS and NK; with or without genetic modifications. In some embodiments, the NK cells intended for expansion include the NK cells in PBMC cultures, UCB cultures, etc. Accordingly, PBMC cultures containing a small population of NK cells may be used, in the method as disclosed herein, for enriching and expanding the NK cells. In an example, the NK cells are enriched from Leukopak. Leukopak is an enriched apheresis product collected via leukapheresis and contains a high concentration of PBMCs. In an embodiment, the PBMCs are obtained from a healthy donor.

The NK cells, in an embodiment herein, may be modified NK cells comprising one or more chimeric antigen receptors (CAR) modifications. In an embodiment, the NK cell is a CAR-NK cell capable of expressing chimeric antigen receptors (CAR). Modification of immune cells, such as NK cells, to enable CAR expression for binding to tumor antigens and inducing tumor cell cytotoxicity is a known strategy in cancer immunotherapy. Typically, CARs are engineered antibody-like molecules that include extracellular domain(s), for antigen binding, expressed as a cell surface receptor, preferably is an antibody derived single chain variable fragment (scFv), for e.g.: derived from anti-CD19, anti-BCMA, anti-CD123, anti-CD138, anti-CD20, anti-CD22, anti-CD38, anti HLA-G, anti PDL1 and anti-CD5 antibodies; hinge domain, for e.g.: derived from CD8, CD28, Ig4, etc.; transmembrane domain, for e.g.: transmembrane domain of CD3z, CD28, etc.; and cytosolic signal transduction domain, for e.g.: human CD3z, having one or more co-stimulatory domains for e.g.: derived from CD28, 41BB, etc. The CAR modification may further include incorporation of one or more transgenes to express cytokines for e.g.: IL-7, IL-12, IL-15, IL-18, IL-21, IL-23, etc. CAR modifications are achieved by introducing, by transfection or viral transduction, CAR transgenes encoding the CAR into the NK cells using gene editing tools and techniques for subsequent expression.

In an exemplary embodiment, the CAR is an anti-CD19 CAR which includes an antigen binding domain of anti-CD19 antibody; a signaling domain of human CD8; a hinge and a transmembrane domain along with the cytosolic domain of human CD32, The CAR modification, according to embodiments herein, further includes introducing transgenes for the expression of co-stimulatory molecule 41BB and secretory IL-15. The expression of 41BB and secretory IL-15 is achieved by including the 41BB gene and secretory IL-15 gene within the CAR transgenes for introduction into NK cells. Accordingly, in an embodiment, the CAR transgene includes a gene encoding antigen binding domain of anti-CD19 antibody; gene encoding the signaling domain of human CD8; a gene encoding hinge, transmembrane domain along and cytosolic domain of human CD32; gene encoding 41BB and secretory IL-15. Plasmids having such CAR transgene are publicly available and may be used suitably in various embodiments herein. For example, FMC63 CAR plasmid obtained from plasmid repository such as Addgene: pRRL.SIN.EFIA.CD19-FMC63.218.CAR-3G, may be used.

In another embodiment, the NK cell comprises a heterologous nucleotide encoding IL-15 within the anti-CD19 CAR transgene. In an embodiment, the nucleotide encoding IL-15 has a sequence as set forth in SEQ ID NO: 1.

### Feeder Layer-Mesenchymal Stem Cells

Embodiments herein include a feeder layer. The term “feeder layer”, as used herein, refers to a layer of cells used for supporting the growth and proliferation of another cell. The feeder layer, according to embodiments herein, refers to mesenchymal stem cells. The mesenchymal stem cells (also referred to herein as “MSC” or “MSCs”), according to embodiments herein, are mammalian, preferably human, mesenchymal stem cells. It refers to MSCs, wherein the major histocompatibility complex (MHC) family of genes are disrupted or deleted. The terms “disrupted”, “deleted”, or “knock out”, used interchangeably herein, refers to genetic modification of one or more genes such that the cell is incapable of expressing the gene product, for e.g.: MHC class I, MHC class II, or both. It refers to partial or complete disruption of a gene by inclusion of one or more mutations, complete or partial deletion of genes, etc. Various gene editing methods are known in the art and may be used in achieving the disruption of the genes according to embodiments herein. In an embodiment, CRISPR/Cas gene editing system is used to disrupt/delete the MHC class I genes and/or MHC class II genes in the MSCs. In an embodiment, the MSCs are having a disrupted or deleted MHC class I gene, also referred to herein as class I null MSCs. In another embodiment, the MSCs are having a disrupted or deleted MHC class II gene, also referred to herein as class II null MSCs. In another embodiment, the MSCs are having a disrupted or deleted MHC class I and MHC class II genes, also referred to herein as class I and class II null MSCs.

Various strategies are known and may be used for disruption of MHC class I/II gene. Disruption of MHC class I/II gene, in accordance with the present disclosure, may be performed by knockout mutations in genes of two key components, viz. β2 Microglobulin (encoded by B2M gene; gene ID: ENSG00000166710; location: Chromosome 15: 44,711,358-44,718,851) of MHC class I genes; and Class II major histocompatibility class transactivator (encoded by CIITA gene; gene ID: ENSG00000179583; location: Chromosome 16: 10,866,222-10,943,021) of MHC class II genes (i.e., human leukocyte antigen [HLA] I/II knockout). It is understood that various methods and tools of gene modifications would be apparent to a person skilled in the art, and maybe used to achieve the MSCs according to embodiments herein, in light of the present disclosure.

In an embodiment, the feeder layer comprises a population of mesenchymal stem cells (MSCs) having disrupted or deleted B2M gene and/or CIITA gene.

The MSCs, according to embodiments herein, are further capable of expressing membrane bound Interleukin 21 (IL-21). Accordingly, in an embodiment, the MSCs further comprise a heterologous gene for expressing IL-21. In an embodiment, the feeder layer comprises a population of mesenchymal stem cells (MSCs) comprising a heterologous gene for expressing IL-21. In an embodiment, the feeder layer comprises MSCs expressing membrane bound Interleukin 21.

It is understood that, in light of the present disclosure, various modifications and approaches can be practiced and would be apparent to a person skilled in the art in achieving MHC Class I/Class II null MSCs in accordance with the present disclosure.

In an embodiment, the heterologous gene encoding IL-21 comprises a nucleotide having a sequence as set forth in SEQ ID NO: 2.

The sequence details, according to the present disclosure, are depicted in Table 1.

TABLE 1

| SEQ ID NO | Description | Nucleotide sequence |
|---|---|---|
| SEQ ID | IL-15 | GGCATACATGTCTTCATATTGGGCTGCTTTAGCGCGGGACTT |
| NO: 1 | | CCAAAGACTGAGGCTAACTGGGTCAATGTGATCAGCGACTT |
| | | GAAGAAGATCGAAGACCTGATTCAGTCTATGCACATAGACG |
| | | CCACTCTTTACACGGAGTCTGATGTGCATCCCAGTTGTAAAG |
| | | TAACGGCGATGAAGTGCTTTCTTCTTGAATTGCAAGTGATCT |
| | | CTCTTGAATCAGGGGATGCAAGTATACATGACACCGTAGAG |
| | | AACCTGATAATACTCGCTAATAACTCATTGTCCTCCAACGG |
| | | AAATGTGACGGAGAGTGGCTGCAAGGAATGCGAGGAACTG |
| | | GAAGAGAAGAACATCAAGGAATTTCTTCAAAGTTTCGTTCA |
| | | TATTGTGCAGATGTTCATCAACACAAGT |
| SEQ ID | IL-21 | >NM_021803.4 Homo sapiens interleukin 21 (IL-21), |
| NO: 2 | | transcript variant 1, mRNA |
| | | CTCAGTCAAGCTGAAGTGAAAACGAGACCAAGGTCTAGCTC |
| | | TACTGTTGGTACTTATGAGATCCAGTCCTGGCAACATGGAG |
| | | AGGATTGTCATCTGTCTGATGGTCATCTTCTTGGGGACACTG |
| | | GTCCACAAATCAAGCTCCCAAGGTCAAGATCGCCACATGAT |
| | | TAGAATGCGTCAACTTATAGATATTGTTGATCAGCTGAAAA |
| | | ATTATGTGAATGACTTGGTCCCTGAATTTCTGCCAGCTCCAG |
| | | AAGATGTAGAGACAAACTGTGAGTGGTCAGCTTTTTCCTGC |
| | | TTTCAGAAGGCCCAACTAAAGTCAGCAAATACAGGAAACA |
| | | ATGAAAGGATAATCAATGTATCAATTAAAAAGCTGAAGAG |
| | | GAAACCACCTTCCACAAATGCAGGGAGAAGACAGAAACAC |
| | | AGACTAACATGCCCTTCATGTGATTCTTATGAGAAAAAACC |
| | | ACCCAAAGAATTCCTAGAAAGATTCAAATCACTTCTCCAAA |
| | | AGATGATTCATCAGCATCTGTCCTCTAGAACACACGGAAGT |
| | | GAAGATTCCTGAGGATCTAACTTGCAGTTGGACACTATGTT |
| | | ACATACTCTAATATAGTAGTGAAAGTCATTTCTTTGTATTCC |
| | | AAGTGGAGGAGTACAATATATTAGCGATGGGAAAAAAAAA |
| | | CTCATAAGTGTGCAAAGTCAGGATTATTTCCCCATAATCACT |
| | | ATACAATAGTCTGATTTCCTATGTTATTGATTTCTATGACTT |
| | | CTGAAGTTTTTACTTTATCTCACTGCCCAACTTTGACATTTCT |
| | | TGGTTGAAGGAGAAGGCTAAAATTTTTAACCAAAAATACAG |
| | | TTTTGACATCTATTGATTTTATATTAACCTTGAGCTTGATTAC |
| | | CACCAGAATGAGGTCTATTATGATCATAACTTAAAACATAT |
| | | ATACCTTCATTCCAGCTGATAATATAGAAGCATAGAAGCAA |
| | | AATATATAAAAGCAAAATGAAAAGTTGACATCTTTATTCTT |
| | | TTAAGGAAATGAAAACTGAGTGTTTAAAAACTTATATCTGA |
| | | ATAGTTTTTTAAATCTCAGAATGTCTAACATATAAATATGTA |
| | | ATTTATTTATGCACCGGGGGATACTAGATGGCTATAAAAGC |
| | | CTTACTTCTTTATTGGGGTTTATAAAAATATTTAAATACTAT |
| | | AGATATGAATGTATTTATGGAATAGACTCTTAGTCAGTGGC |
| | | AATAGAGTATTAAGAAGTGGATTTAATATTACCTTCCTCCA |
| | | GCCCCCAGTCCCCTTATAAGCTAACATTTTAAGCTTGTGGAA |
| | | ATGGCAATTTAAACCTATAGAGGGACATATGCCAGGTTGTC |
| | | TTCTGGAACCAAACATAGTTTTAAAAGTATTGTAGCTCTCTT |
| | | CTCAAACTGTTATAATGATCATATTATGAGCAAGCTAGTAA |
| | | CTCCAAATATTTGTCTCCTTCTTTCTTCTTTCGTTTCTTTTCTT |
| | | CCTTCTTTTTAAAATGAAACTTCTGCTTTGACAAAATTTCAC |
| | | AACATGTTTAACATTTTGGACACTTATAGTTACTATACTTTG |
| | | GATAAGACAAATTCAATCACCTATATAATTTAAAAATCAGG |
| | | AGACAAACCAAGAGGCCAGGACTCCTGTCCAAGTTGAAAA |
| | | CAGACTTTCGCATAAGCATTCCAATCTTTCAGACTTGGGTAG |
| | | TAAAAACAAGCAACCTACTCTACCTGCCATTTCATATCAACT |
| | | CCAGCACAGCTTGTTTAAACATGAAGTTAAAAACTCTAGAA |
| | | AAGTAAAATGTTCGCTAGAATGGTATAAGCTATTTGAGAAT |
| | | CTCTTATTGTTAAAATAGTTGTGATTATGCAAGAGGATGTGG |
| | | GATGAGAAATGCTTTATGCTCTGAGAAGAAAAATTATGTGC |
| | | TTAAAACAAATACAGACTTAACCTACAGTTCTAAAAGATAT |
| | | ATTGAGAATTATTTAATCAAATCTCAATGAAATGTTTTATCA |
| | | TCAGCAAGTAGACTCAGAAAAAAGAAAAAAAAATTTATCA |
| | | TCAGAAACAAGGTACTCTGTTTTAACATTTTAGAACATGTAT |
| | | GACAAAGGAATTATGTGCCTGATGATGATAATATATATCTA |
| | | AAATGTGCCTCAGTTTCAAATACGCCAGCCCGGGTTGTTGT |
| | | GACAAAATGTGGAAGCAGGCACAAAGATAGTGAGAAAGAT |
| | | GGAATCTGTATTTCTTGTTTCTGAATGTATAGCTATTAACTT |
| | | CTTGGATATTGTATTTCATATTTAAAATAATGTATTTCTTTTG |
| | | GCATGTTTATTGTTAAAAATTTCTGTTCCAATTATTTTGATC |
| | | AGGGTCTCTATCTTAAAAGTAAAAATAACTCAGTGAGGGTT |
| | | TGGCTAAGAGCCTTCATTAAACAGAGTTTAAACACCCAGAT |
| | | AAACTCTGCTGTTTAACCAGAGTTAAACCCACCAGCACTCC |
| | | AGGAAAGGCAATGCATTGGAATAAACAAGATGAAGCAGTG |
| | | AGACTTGAACAGCTCCATCTTCACTGCTCTATGTTCAAGATG |
| | | GCCAGTTCTTAAAAGGAATATAAAGATAACCCATAAATATA |
| | | ACTGTTCATCTATGGACAGTAATACAGGAGACGTGAAATTT |
| | | TGTTGACTATGAAATAGTGGTCACTGTTTAAACTCTTTGGGT |
| | | TTCCATTTCTTTATCTGTAGAATGAGAGTGCTGGACTTAATC |
| | | TCTAATGTCTATTCTTTCTTTTTTAAAGCTTTAAATGTCTATG |
| | | AATATCTAAAGTTCTGGGTATTCTTACAGAAACATCTGATCA |
| | | TACTTCTGAGTAAAACTTTCTACTGAGTCTGATGTTCAGTTG |
| | | CTCAGTATTATTTCAATAACATTTGTAGCAACACTCACTGCA |
| | | GGAACAAGGGACATATTCCAAGAAAAAAAAGGAAGGAAGG |
| | | AAGGAGGGGAGAAAAGGAAACATTATTATAGGTCATGAGA |
| | | GTTCAGGGAAAATTAATCATGATCTTTATTATTTTAGTTCAA |
| | | AGTGTCTTCTTGGAACACTTGTATAAAATTGTAGCATTATTA |
| | | TTCTATATTATTGCTCTGTTGTATTTACATATGCATCTGAGA |
| | | ATTTAGCTAATATGAACTATGTAGTTTATAACTTAATAATTA |
| | | TTTATTATATATTTGATTTTAAATGTTCATGTTTATGGCTTCT |
| | | TATTTAAGACCTGATCATATTAAATACTACCCGCCCAGTA |
| SEQ ID | SV40LT | ATGGATAAAGTTTTAAACAGAGAGGAATCTTTGCAGCTAAT |
| NO: 3 | | GGACCTTCTAGGTCTTGAAAGGAGTGCCTGGGGGAATATTC |
| | | CTCTGATGAGAAAGGCATATTTAAAAAAATGCAAGGAGTTT |
| | | CATCCTGATAAAGGAGGAGATGAAGAAAAAATGAAGAAAA |
| | | TGAATACTCTGTACAAGAAAATGGAAGATGGAGTAAAATAT |
| | | GCTCATCAACCTGACTTTGGAGGCTTCTGGGATGCAACTGA |
| | | GATTCCAACCTATGGAACTGATGAATGGGAGCAGTGGTGGA |
| | | ATGCCTTTAATGAGGAAAACCTGTTTTGCTCAGAAGAAATG |
| | | CCATCTAGTGATGATGAGGCTACTGCTGACTCTCAACATTCT |
| | | ACTCCTCCAAAAAAGAAGAGAAAGGTAGAAGACCCCAAGG |
| | | ACTTTCCTTCAGAATTGCTAAGTTTTTTGAGTCATGCTGTGT |
| | | TTAGTAATAGAACTCTTGCTTGCTTTGCTATTTACACCACAA |
| | | AGGAAAAAGCTGCACTGCTATACAAGAAAATTATGGAAAA |
| | | ATATTCTGTAACCTTTATAAGTAGGCATAACAGTTATAATCA |
| | | TAACATACTGTTTTTTCTTACTCCACACAGGCATAGAGTGTC |
| | | TGCTATTAATAACTATGCTCAAAAATTGTGTACCTTTAGCTT |
| | | TTTAATTTGTAAAGGGGTTAATAAGGAATATTTGATGTATA |
| | | GTGCCTTGACTAGAGATCCATTTTCTGTTATTGAGGAAAGTT |
| | | TGCCAGGTGGGTTAAAGGAGCATGATTTTAATCCAGAAGAA |
| | | GCAGAGGAAACTAAACAAGTGTCCTGGAAGCTTGTAACAG |
| | | AGTATGCAATGGAAACAAAATGTGATGATGTGTTGTTATTG |
| | | CTTGGGATGTACTTGGAATTTCAGTACAGTTTTGAAATGTGT |
| | | TTAAAATGTATTAAAAAAGAACAGCCCAGCCACTATAAGTA |
| | | CCATGAAAAGCATTATGCAAATGCTGCTATATTTGCTGACA |
| | | GCAAAAACCAAAAAACCATATGCCAACAGGCTGTTGATACT |
| | | GTTTTAGCTAAAAAGCGGGTTGATAGCCTACAATTAACTAG |
| | | AGAACAAATGTTAACAAACAGATTTAATGATCTTTTGGATA |
| | | GGATGGATATAATGTTTGGTTCTACAGGCTCTGCTGACATA |
| | | GAAGAATGGATGGCTGGAGTTGCTTGGCTACACTGTTTGTT |
| | | GCCCAAAATGGATTCAGTGGTGTATGACTTTTTAAAATGCA |
| | | TGGTGTACAACATTCCTAAAAAAAGATACTGGCTGTTTAAA |
| | | GGACCAATTGATAGTGGTAAAACTACATTAGCAGCTGCTTT |
| | | GCTTGAATTATGTGGGGGGAAAGCTTTAAATGTTAATTTGC |
| | | CCTTGGACAGGCTGAACTTTGAGCTAGGAGTAGCTATTGAC |
| | | CAGTTTTTAGTAGTTTTTGAGGATGTAAAGGGCACTGGAGG |
| | | GGAGTCCAGAGATTTGCCTTCAGGTCAGGGAATTAATAACC |
| | | TGGACAATTTAAGGGATTATTTGGATGGCAGTGTTAAGGTA |
| | | AACTTAGAAAAGAAACACCTAAATAAAAGAACTCAAATATT |
| | | TCCCCCTGGAATAGTCACCATGAATGAGTACAGTGTGCCTA |
| | | AAACACTGCAGGCCAGATTTGTAAAACAAATAGATTTTAGG |
| | | CCCAAAGATTATTTAAAGCATTGCCTGGAACGCAGTGAGTT |
| | | TTTGTTAGAAAAGAGAATAATTCAAAGTGGCATTGCTTTGC |
| | | TTCTTATGTTAATTTGGTACAGACCTGTGGCTGAGTTTGCTC |
| | | AAAGTATTCAGAGCAGAATTGTGGAGTGGAAAGAGAGATT |
| | | GGACAAAGAGTTTAGTTTGTCAGTGTATCAAAAAATGAAGT |
| | | TTAATGTGGCTATGGGAATTGGAGTTTTAGATTGGCTAAGA |
| | | AACAGTGATGATGATGATGAAGACAGCCAGGAAAATGCTG |
| | | ATAAAAATGAAGATGGTGGGGAGAAGAACATGGAAGACTC |
| | | AGGGCATGAAACAGGCATTGATTCACAGTCCCAAGGCTCAT |
| | | TTCAGGCCCCTCAGTCCTCACAGTCTGTTCATGATCATAATC |
| | | AGCCATACCACATTTGTAGAGGTTTTACTTGCTTTAAAAAAC |
| | | CTCCCACACCTCCCCCTGAACCTGAAACATAA |
| SEQ ID | hTERT | hTERT [NM_198253.3] locus 1601-4996 |
| NO: 4 | | ATGCCGCGCGCTCCCCGCTGCCGAGCCGTGCGCTCCCTGCT |
| | | GCGCAGCCACTACCGCGAGGTGCTGCCGCTGGCCACGTTCG |
| | | TGCGGCGCCTGGGGCCCCAGGGCTGGCGGCTGGTGCAGCGC |
| | | GGGGACCCGGCGGCTTTCCGCGCGCTGGTGGCCCAGTGCCT |
| | | GGTGTGCGTGCCCTGGGACGCACGGCCGCCCCCCGCCGCCC |
| | | CCTCCTTCCGCCAGGTGTCCTGCCTGAAGGAGCTGGTGGCC |
| | | CGAGTGCTGCAGAGGCTGTGCGAGCGCGGCGCGAAGAACG |
| | | TGCTGGCCTTCGGCTTCGCGCTGCTGGACGGGGCCCGCGGG |
| | | GGCCCCCCCGAGGCCTTCACCACCAGCGTGCGCAGCTACCT |
| | | GCCCAACACGGTGACCGACGCACTGCGGGGGAGCGGGGCG |
| | | TGGGGGCTGCTGCTGCGCCGCGTGGGCGACGACGTGCTGGT |
| | | TCACCTGCTGGCACGCTGCGCGCTCTTTGTGCTGGTGGCTCC |
| | | CAGCTGCGCCTACCAGGTGTGCGGGCCGCCGCTGTACCAGC |
| | | TCGGCGCTGCCACTCAGGCCCGGCCCCCGCCACACGCTAGT |
| | | GGACCCCGAAGGCGTCTGGGATGCGAACGGGCCTGGAACC |
| | | ATAGCGTCAGGGAGGCCGGGGTCCCCCTGGGCCTGCCAGCC |
| | | CCGGGTGCGAGGAGGCGCGGGGGCAGTGCCAGCCGAAGTC |
| | | TGCCGTTGCCCAAGAGGCCCAGGCGTGGCGCTGCCCCTGAG |
| | | CCGGAGCGGACGCCCGTTGGGCAGGGGTCCTGGGCCCACCC |
| | | GGGCAGGACGCGTGGACCGAGTGACCGTGGTTTCTGTGTGG |
| | | TGTCACCTGCCAGACCCGCCGAAGAAGCCACCTCTTTGGAG |
| | | GGTGCGCTCTCTGGCACGCGCCACTCCCACCCATCCGTGGG |
| | | CCGCCAGCACCACGCGGGCCCCCCATCCACATCGCGGCCAC |
| | | CACGTCCCTGGGACACGCCTTGTCCCCCGGTGTACGCCGAG |
| | | ACCAAGCACTTCCTCTACTCCTCAGGCGACAAGGAGCAGCT |
| | | GCGGCCCTCCTTCCTACTCAGCTCTCTGAGGCCCAGCCTGAC |
| | | TGGCGCTCGGAGGCTCGTGGAGACCATCTTTCTGGGTTCCA |
| | | GGCCCTGGATGCCAGGGACTCCCCGCAGGTTGCCCCGCCTG |
| | | CCCCAGCGCTACTGGCAAATGCGGCCCCTGTTTCTGGAGCT |
| | | GCTTGGGAACCACGCGCAGTGCCCCTACGGGGTGCTCCTCA |
| | | AGACGCACTGCCCGCTGCGAGCTGCGGTCACCCCAGCAGCC |
| | | GGTGTCTGTGCCCGGGAGAAGCCCCAGGGCTCTGTGGCGGC |
| | | CCCCGAGGAGGAGGACACAGACCCCCGTCGCCTGGTGCAGC |
| | | TGCTCCGCCAGCACAGCAGCCCCTGGCAGGTGTACGGCTTC |
| | | GTGCGGGCCTGCCTGCGCCGGCTGGTGCCCCCAGGCCTCTG |
| | | GGGCTCCAGGCACAACGAACGCCGCTTCCTCAGGAACACCA |
| | | AGAAGTTCATCTCCCTGGGGAAGCATGCCAAGCTCTCGCTG |
| | | CAGGAGCTGACGTGGAAGATGAGCGTGCGGGACTGCGCTTG |
| | | GCTGCGCAGGAGCCCAGGGGTTGGCTGTGTTCCGGCCGCAG |
| | | AGCACCGTCTGCGTGAGGAGATCCTGGCCAAGTTCCTGCAC |
| | | TGGCTGATGAGTGTGTACGTCGTCGAGCTGCTCAGGTCTTTC |
| | | TTTTATGTCACGGAGACCACGTTTCAAAAGAACAGGCTCTTT |
| | | TTCTACCGGAAGAGTGTCTGGAGCAAGTTGCAAAGCATTGG |
| | | AATCAGACAGCACTTGAAGAGGGTGCAGCTGCGGGAGCTGT |
| | | CGGAAGCAGAGGTCAGGCAGCATCGGGAAGCCAGGCCCGC |
| | | CCTGCTACGTCCAGACTCCGCTTCATCCCCAAGCCTGACGG |
| | | GCTGCGGCCGATTGTGAACATGGACTACGTCGTGGGAGCCA |
| | | GAACGTTCCGCAGAGAAAAGAGGGCCGAGCGTCTCACCTCG |
| | | AGGGTGAAGGCACTGTTCAGCGTGCTCAACTACGAGCGGGC |
| | | GCGGCGCCCCGGCCTCCTGGGCGCCTCTGTGCTGGGCCTGG |
| | | ACGATATCCACAGGGCCTGGCGCACCTTCGTGCTGCGTGTG |
| | | CGGGCCCAGGACCCGCCGCCTGAGCTGTACTTTGTCAAGGT |
| | | GGATGTGACGGGCGCGTACGACACCATCCCCCAGGACAGGC |
| | | TCACGGAGGTCATCGCCAGCATCATCAAACCCCAGAACACG |
| | | TACTGCGTGCGTCGGTATGCCGTGGTCCAGAAGGCCGCCCA |
| | | TGGGCACGTCCGCAAGGCCTTCAAGAGCCACGTCTCTACCT |
| | | TGACAGACCTCCAGCCGTACATGCGACAGTTCGTGGCTCAC |
| | | CTGCAGGAGACCAGCCCGCTGAGGGATGCCGTCGTCATCGA |
| | | GCAGAGCTCCTCCCTGAATGAGGCCAGCAGTGGCCTCTTCG |
| | | ACGTCTTCCTACGCTTCATGTGCCACCACGCCGTGCGCATCA |
| | | GGGGCAAGTCCTACGTCCAGTGCCAGGGGATCCCGCAGGGC |
| | | TCCATCCTCTCCACGCTGCTCTGCAGCCTGTGCTACGGCGAC |
| | | ATGGAGAACAAGCTGTTTGCGGGGATTCGGCGGGACGGGCT |
| | | GCTCCTGCGTTTGGTGGATGATTTCTTGTTGGTGACACCTCA |
| | | CCTCACCCACGCGAAAACCTTCCTCAGGACCCTGGTCCGAG |
| | | GTGTCCCTGAGTATGGCTGCGTGGTGAACTTGCGGAAGACA |
| | | GTGGTGAACTTCCCTGTAGAAGACGAGGCCCTGGGTGGCAC |
| | | GGCTTTTGTTCAGATGCCGGCCCACGGCCTATTCCCCTGGTG |
| | | CGGCCTGCTGCTGGATACCCGGACCCTGGAGGTGCAGAGCG |
| | | ACTACTCCAGCTATGCCCGGACCTCCATCAGAGCCAGTCTC |
| | | ACCTTCAACCGCGGCTTCAAGGCTGGGAGGAACATGCGTCG |
| | | CAAACTCTTTGGGGTCTTGCGGCTGAAGTGTCACAGCCTGTT |
| | | TCTGGATTTGCAGGTGAACAGCCTCCAGACGGTGTGCACCA |
| | | ACATCTACAAGATCCTCCTGCTGCAGGCGTACAGGTTTCAC |
| | | GCATGTGTGCTGCAGCTCCCATTTCATCAGCAAGTTTGGAA |
| | | GAACCCCACATTTTTCCTGCGCGTCATCTCTGACACGGCCTC |
| | | CCTCTGCTACTCCATCCTGAAAGCCAAGAACGCAGGGATGT |
| | | CGCTGGGGGCCAAGGGCGCCGCCGGCCCTCTGCCCTCCGAG |
| | | GCCGTGCAGTGGCTGTGCCACCAAGCATTCCTGCTCAAGCT |
| | | GACTCGACACCGTGTCACCTACGTGCCACTCCTGGGGTCAC |
| | | TCAGGACAGCCCAGACGCAGCTGAGTCGGAAGCTCCCGGG |
| | | GACGACGCTGACTGCCCTGGAGGCCGCAGCCAACCCGGCAC |
| | | TGCCCTCAGACTTCAAGACCATCCTGGACAGGCACAACGAA |
| | | CGCCGCTTCCTCAGGAACACCAAGAAGTTCATCTCCCTGGG |
| | | GAAGCATGCCAAGCTCTCGCTGCAGGAGCTGACGTGGAAGA |
| | | TGAGCGTGCGGGACTGCGCTTGGCTGCGCAGGAGCCCAGGG |
| | | GTTGGCTGTGTTCCGGCCGCAGAGCACCGTCTGCGTGAGGA |
| | | GATCCTGGCCAAGTTCCTGCACTGGCTGATGAGTGTGTACG |
| | | TCGTCGAGCTGCTCAGGTCTTTCTTTTATGTCACGGAGACCA |
| | | CGTTTCAAAAGAACAGGCTCTTTTTCTACCGGAAGAGTGTCT |
| | | GGAGCAAGTTGCAAAGCATTGGAATCAGACAGCACTTGAA |
| | | GAGGGTGCAGCTGCGGGAGCTGTCGGAAGCAGAGGTCAGG |
| | | CAGCATCGGGAAGCCAGGCCCGCCCTGCTGACGTCCAGACT |
| | | CCGCTTCATCCCCAAGCCTGACGGGCTGCGGCCGATTGTGA |
| | | ACATGGACTACGTCGTGGGAGCCAGAACGTTCCGCAGAGAA |
| | | AAGAGGGCCGAGCGTCTCACCTCGAGGGTGAAGGCACTGTT |
| | | CAGCGTGCTCAACTACGAGCGGGCGCGGCGCCCCGGCCTCC |
| | | TGGGCGCCTCTGTGCTGGGCCTGGACGATATCCACAGGGCC |
| | | TGGCGCACCTTCGTGCTGCGTGTGCGGGCCCAGGACCCGCC |
| | | GCCTGAGCTGTACTTTGTCAAGGTGGATGTGACGGGCGCGT |
| | | ACGACACCATCCCCCAGGACAGGCTCACGGAGGTCATCGCC |
| | | AGCATCATCAAACCCCAGAACACGTACTGCGTGCGTCGGTA |
| | | TGCCGTGGTCCAGAAGGCCGCCCATGGGCACGTCCGCAAGG |
| | | CCTTCAAGAGCCACGTCTCTACCTTGACAGACCTCCAGCCGT |
| | | ACATGCGACAGTTCGTGGCTCACCTGCAGGAGACCAGCCCG |
| | | CTGAGGGATGCCGTCGTCATCGAGCAGAGCTCCTCCCTGAA |
| | | TGAGGCCAGCAGTGGCCTCTTCGACGTCTTCCTACGCTTCAT |
| | | GTGCCACCACGCCGTGCGCATCAGGGGCAAGTCCTACGTCC |
| | | AGTGCCAGGGGATCCCGCAGGGCTCCATCCTCTCCACGCTG |
| | | CTCTGCAGCCTGTGCTACGGCGACATGGAGAACAAGCTGTT |
| | | TGCGGGGATTCGGCGGGACGGGCTGCTCCTGCGTTTGGTGG |
| | | ATGATTTCTTGTTGGTGACACCTCACCTCACCCACGCGAAAA |
| | | CCTTCCTCAGGACCCTGGTCCGAGGTGTCCCTGAGTATGGCT |
| | | GCGTGGTGAACTTGCGGAAGACAGTGGTGAACTTCCCTGTA |
| | | GAAGACGAGGCCCTGGGTGGCACGGCTTTTGTTCAGATGCC |
| | | GGCCCACGGCCTATTCCCCTGGTGCGGCCTGCTGCTGGATA |
| | | CCCGGACCCTGGAGGTGCAGAGCGACTACTCCAGCTATGCC |
| | | CGGACCTCCATCAGAGCCAGTCTCACCTTCAACCGCGGCTT |
| | | CAAGGCTGGGAGGAACATGCGTCGCAAACTCTTTGGGGTCT |
| | | TGCGGCTGAAGTGTCACAGCCTGTTTCTGGATTTGCAGGTG |
| | | AACAGCCTCCAGACGGTGTGCACCAACATCTACAAGATCCT |
| | | CCTGCTGCAGGCGTACAGGTTTCACGCATGTGTGCTGCAGC |
| | | TCCCATTTCATCAGCAAGTTTGGAAGAACCCCACATTTTTCC |
| | | TGCGCGTCATCTCTGACACGGCCTCCCTCTGCTACTCCATCC |
| | | TGAAAGCCAAGAACGCAGGGATGTCGCTGGGGGCCAAGGG |
| | | CGCCGCCGGCCCTCTGCCCTCCGAGGCCGTGCAGTGGCTGT |
| | | GCCACCAAGCATTCCTGCTCAAGCTGACTCGACACCGTGTC |
| | | ACCTACGTGCCACTCCTGGGGTCACTCAGGACAGCCCAGAC |
| | | GCAGCTGAGTCGGAAGCTCCCGGGGACGACGCTGACTGCCC |
| | | TGGAGGCCGCAGCCAACCCGGCACTGCCCTCAGACTTCAAG |
| | | ACCATCCTGGAC |
| SEQ ID | CD19 | >NM_001178098.2 Homo sapiens CD19 molecule (CD19), |
| NO: 5 | | transcript variant 1, mRNA |
| | | AAGCTGGGTGCCCCGGAGAGTCTGACCACCATGCCACCTCC |
| | | TCGCCTCCTCTTCTTCCTCCTCTTCCTCACCCCCATGGAAGTC |
| | | AGGCCCGAGGAACCTCTAGTGGTGAAGGTGGAAGAGGGAG |
| | | ATAACGCTGTGCTGCAGTGCCTCAAGGGGACCTCAGATGGC |
| | | CCCACTCAGCAGCTGACCTGGTCTCGGGAGTCCCCGCTTAA |
| | | ACCCTTCTTAAAACTCAGCCTGGGGCTGCCAGGCCTGGGAA |
| | | TCCACATGAGGCCCCTGGCCATCTGGCTTTTCATCTTCAACG |
| | | TCTCTCAACAGATGGGGGGCTTCTACCTGTGCCAGCCGGGG |
| | | CCCCCCTCTGAGAAGGCCTGGCAGCCTGGCTGGACAGTCAA |
| | | TGTGGAGGGCAGCGGGGAGCTGTTCCGGTGGAATGTTTCGG |
| | | ACCTAGGTGGCCTGGGCTGTGGCCTGAAGAACAGGTCCTCA |
| | | GAGGGCCCCAGCTCCCCTTCCGGGAAGCTCATGAGCCCCAA |
| | | GCTGTATGTGTGGGCCAAAGACCGCCCTGAGATCTGGGAGG |
| | | GAGAGCCTCCGTGTCTCCCACCGAGGGACAGCCTGAACCAG |
| | | AGCCTCAGCCAGGACCTCACCATGGCCCCTGGCTCCACACT |
| | | CTGGCTGTCCTGTGGGGTACCCCCTGACTCTGTGTCCAGGGG |
| | | CCCCCTCTCCTGGACCCATGTGCACCCCAAGGGGCCTAAGT |
| | | CATTGCTGAGCCTAGAGCTGAAGGACGATCGCCCGGCCAGA |
| | | GATATGTGGGTAATGGAGACGGGTCTGTTGTTGCCCCGGGC |
| | | CACAGCTCAAGACGCTGGAAAGTATTATTGTCACCGTGGCA |
| | | ACCTGACCATGTCATTCCACCTGGAGATCACTGCTCGGCCA |
| | | GTACTATGGCACTGGCTGCTGAGGACTGGTGGCTGGAAGGT |
| | | CTCAGCTGTGACTTTGGCTTATCTGATCTTCTGCCTGTGTTCC |
| | | CTTGTGGGCATTCTTCATCTTCAAAGAGCCCTGGTCCTGAGG |
| | | AGGAAAAGAAAGCGAATGACTGACCCCACCAGGAGATTCT |
| | | TCAAAGTGACGCCTCCCCCAGGAAGCGGGCCCCAGAACCAG |
| | | TACGGGAACGTGCTGTCTCTCCCCACACCCACCTCAGGCCTC |
| | | GGACGCGCCCAGCGTTGGGCCGCAGGCCTGGGGGGCACTGC |
| | | CCCGTCTTATGGAAACCCGAGCAGCGACGTCCAGGCGGATG |
| | | GAGCCTTGGGGTCCCGGAGCCCGCCGGGAGTGGGCCCAGA |
| | | AGAAGAGGAAGGGGAGGGCTATGAGGAACCTGACAGTGAG |
| | | GAGGACTCCGAGTTCTATGAGAACGACTCCAACCTTGGGCA |
| | | GGACCAGCTCTCCCAGGATGGCAGCGGCTACGAGAACCCTG |
| | | AGGATGAGCCCCTGGGTCCTGAGGATGAAGACTCCTTCTCC |
| | | AACGCTGAGTCTTATGAGAACGAGGATGAAGAGCTGACCCA |
| | | GCCGGTCGCCAGGACAATGGACTTCCTGAGCCCTCATGGGT |
| | | CAGCCTGGGACCCCAGCCGGGAAGCAACCTCCCTGGCAGGG |
| | | TCCCAGTCCTATGAGGATATGAGAGGAATCCTGTATGCAGC |
| | | CCCCCAGCTCCGCTCCATTCGGGGCCAGCCTGGACCCAATC |
| | | ATGAGGAAGATGCAGACTCTTATGAGAACATGGATAATCCC |
| | | GATGGGCCAGACCCAGCCTGGGGAGGAGGGGGCCGCATGG |
| | | GCACCTGGAGCACCAGGTGATCCTCAGGTGGCCAGCCTGGA |
| | | TCTCCTCAAGTCCCCAAGATTCACACCTGACTCTGAAATCTG |
| | | AAGACCTCGAGCAGATGATGCCAACCTCTGGAGCAATGTTG |
| | | CTTAGGATGTGTGCATGTGTGTAAGTGTGTGTGTGTGTGTGT |
| | | GTGTGTATACATGCCAGTGACACTTCCAGTCCCCTTTGTATT |
| | | CCTTAAATAAACTCAATGAGCTCTTCCAACCTCCCAGGCAG |
| | | CTCACAGTG |

In other embodiments, the MSC may additionally include heterologous gene for expressing other cytokines for facilitating expansion of NK cells.

The MSC feeder cell layer, according to the present disclosure, may be achieved using gene editing methods generally known in the art. The feeder layer may be maintained in MSC expansion media (for e.g.: StemXVivo® MSC Expansion Media) and passaged at 70-80% confluency with any commercially available cell dissociation reagent. Passaged cells may be cryopreserved using cryomedia, to generate a master cell bank (MCB), or further used in NK cell expansion.

The MSCs for use in the feeder layer, according to embodiments herein, may be obtained from commercial sources or, alternatively, derived from various platforms including induced pluripotent stem cells (iPSCs). In some embodiments, the MSCs are immortalized MSCs. As described previously herein, immortalization of cells, by genetic modification, is performed in order to achieve cells having the ability to proliferate indefinitely, thus facilitating maintenance of the cells for extended duration of time. Various strategies for immortalizing cells are known in the art and may be used in embodiments herein. In an embodiment, the MSCs are immortalized MSCs comprising a SV40LT gene and hTERT gene. In an embodiment, the MSCs are conditionally immortalized cells comprising SV40LT gene and hTERT gene, wherein the expression is inducible by exposure to an inducer, for e.g.: doxycycline. In an embodiment, the SV40LT gene comprises a nucleotide having a sequence as set forth in SEQ ID NO: 3. In an embodiment, the hTERT gene comprises a nucleotide having a sequence as set forth in SEQ ID NO: 4. Accordingly, in an embodiment, the MSC comprises a heterologous nucleotide having a sequence as set forth in SEQ ID NO: 3 and SEQ ID NO: 4. In an embodiment, the feeder layer comprises MSCs expressing membrane bound Interleukin 21, SV40LT gene and hTERT gene.

Various methods are known for immortalization of MSCs. In an example, the MSCs are transfected with a vector construct comprising viral oncogene such as SV40LT, and hTERT to induce immortalization in cells. In another embodiment, the MSCs are transfected with a transposon-based gene expression system, comprising SV40LT and hTERT gene. The transposon gene expression vector for expressing the hTERT and SV40LT antigen and the neomycin-resistant gene may be co-transfected into the cell along with a transposase vector using electroporation. Transfected cells may be cultured with G418 for 10-14 days. Colonies of selected cells may then be cultured with doxycycline continuously to induce immortalization. MSCs expressing SV40LT and hTERT genes may be expanded and frozen using cryomedia to generate the immortalized MSC cell bank. In an embodiment, immortalization is induced in the MSCs comprising SV40LT and hTERT genes by culturing it with an expansion media comprising doxycycline.

In an embodiment, the MSC is derived from iPSCs. iPSCs may be such as those derived using the Yamanaka method (for e.g.: Takahashi K and Yamanaka S Induction of pluripotent stem cells from mouse embryonic and adult fibroblast cultures by defined factors. Cell 126, 663 676 (2006); Takahashi K, Tanabe K, Ohnuki M, Narita M, Ichisaka T, Tomoda K, and Yamanaka S Induction of pluripotent stem cells from adult human fibroblasts by defined factors. Cell 131, 861 872 (2007). In another embodiment, the MSC is derived from engineered iPSCs having disrupted or deleted MHC class I genes and/or MHC class II genes. In yet another embodiment, the MSC is derived from iPSC having disrupted or deleted MHC class I genes and/or MHC class II genes; and a heterologous gene encoding IL-21.

The MSCs, according to embodiments herein, comprise disrupted or deleted major histocompatibility complex (MHC) family of genes. The knock-out of these MHC genes may be performed in the MSCs for e.g.: of commercially obtained cell lines; or in iPSCs such that the MSCs derived from the iPSCs are MHC class I and/or class II null MSCs. Accordingly, in an embodiment, the iPSCs are MHC class I genes and/or MHC class II gene null iPSCs, wherein the iPSCs have a disrupted or deleted MHC class I genes and/or MHC class II genes.

Accordingly, the mesenchymal stem cells are immortalized mesenchymal stem cells obtained by a process selected from: (a) providing a population of induced Pluripotent stem cells (iPSCs) having disrupted or deleted MHC class I gene and/or MHC class II gene and a heterologous gene for expressing IL-21; introducing a heterologous gene to the iPSCs for conditionally immortalizing iPSCs; and culturing the iPSCs in a differentiating medium to obtain the MSCs; or (b) providing a population of induced Pluripotent stem cells (iPSCs) having disrupted or deleted MHC class I gene and/or MHC class II gene and a heterologous gene for expressing IL-21; culturing the iPSCs in a differentiating medium to obtain the MSCs; and introducing a heterologous gene to the MSCs for conditionally immortalizing MSCs. In an embodiment, the iPSCs for preparing MSCs feeder layer comprises disrupted or deleted MHC class I gene and/or MHC class II gene and a heterologous gene having a nucleotide of sequence as set forth in SEQ ID NO: 2. In an example, the differentiating medium is DMEM low glucose medium comprising about 10% FBS, L-Glutamine, iPSC derived MSCs, for use in various embodiments herein, may be obtained by methods generally known in the art. For example, as described in Thanaskody K, Jusop A S, Tye G J, Wan Kamarul Zaman W S, Dass S A, Nordin F. MSCs vs. iPSCs: Potential in therapeutic applications. Front Cell Dev Biol. 2022 Nov. 2; 10:1005926. doi: 10.3389/fcell.2022.1005926. PMID: 36407112; PMCID: PMC9666898.

As described previously herein, disruption of MHC class I/II gene may be achieved by disruption of target sequences on B2M and/or CIITA gene in the iPSCs.

Various differentiation media for achieving differentiation of iPSCs to MSCs are known and may be used herein. To support the expansion of the NK cells or CAR-NK cells, the MSC feeder cells are cultured without Doxycycline before plating the NK cells using the same mesenchymal stem cell culture media. The NK cells or CAR-NK cells may be expanded and maintained using a medium comprising the feeder cells, commercially available NK media along with commercially available IL-2 or IL-2 derivative, as described herein above, to obtain expanded NK cells.

### Method

### Expansion of NK Cells

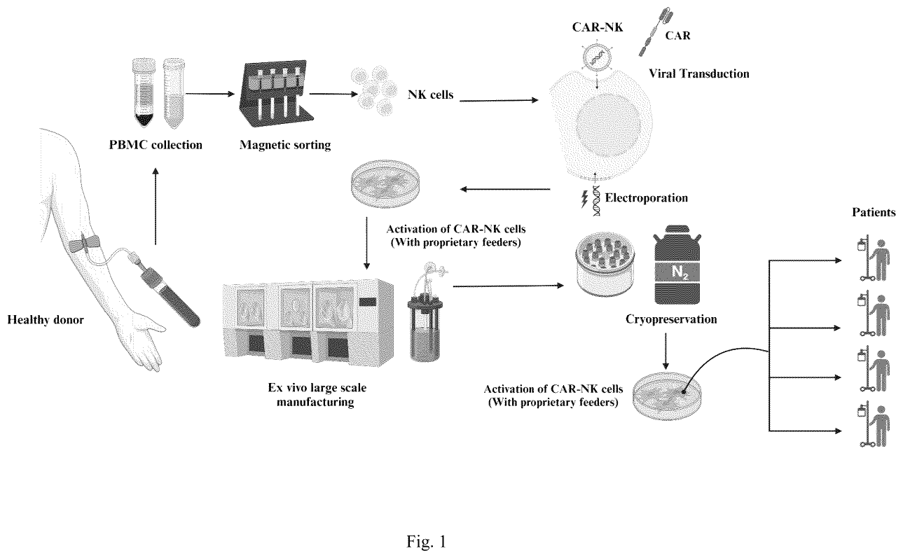

Embodiments herein provide a method for expansion of natural killer cells. In an embodiment, the method for expansion of natural killer (NK) cells comprises culturing the NK cells in the presence of a feeder layer comprising a population of mesenchymal stem cells (MSCs) comprising disrupted or deleted MHC class I gene and/or MHC class II gene and a heterologous gene for expressing Interleukin-21 (IL-21) while providing IL-2 extraneously; and expanding the NK cells by repeatedly replacing the feeder layer and continue culturing the NK cells, to obtain expanded NK cells. FIG. 1 is a schematic representation depicting the method for expansion of NK or CAR-NK cells, in accordance with the embodiments herein.

In an embodiment, the method for expansion of natural killer (NK) cells comprises: (i) obtaining a first population of NK cells from peripheral blood mononuclear cells (PBMCs); (ii) culturing the cells in a medium to obtain activated NK cells, wherein the medium comprises (a) a feeder layer having a population of mesenchymal stem cells (MSCs) comprising disrupted or deleted MHC class I gene and/or MHC class II gene and a heterologous gene for expressing Interleukin-21 (IL-21), and (b) an effective amount of IL-2; and (iii) expanding the activated NK cells by repeatedly replacing the feeder layer and culturing the NK cells, to obtain expanded NK cells. In an embodiment, the first population of NK cells from PBMC is obtained using Magnetic activated cell sorting (MACS). For example, MACS™ available from Miltenyi Biotec may be used to enrich/isolate NK cells from PBMCs.

The term “first population”, as used herein, refers to an initial population or initial number of cells which are intended for expansion. The first population, according to embodiments herein, may be a population of NK cells or CAR-NK cells intended for expansion. In an embodiment, initial population refers to an enriched population of NK cells. Any of these cells may be expanded and maintained using the method as disclosed herein. The term “expanded” or “expanded population”, as used herein, refers to an increased population of cells, wherein the number of cells is higher as compared to the first population. It refers to a multiplied cell population. Accordingly, in an embodiment herein, the expanded population refers to about 100 to 1000 or about 900 to 1000, or greater, fold expansion of cells as compared to the initial or first population of cells.

The method, according to embodiments herein, includes a step of obtaining the NK cells. The NK cells, as described previously herein, may be modified or unmodified NK cells, or CAR-NK cells. Accordingly, disclosed herein, is a method for expansion of NK cells and CAR-NK cells.

The method further includes a step of culturing the NK cells in a medium. Culturing, according to embodiments herein, refers to growing cells by providing the physical conditions (for e.g.: temperature) and chemical conditions (for e.g.: buffers, growth factors, nutrients, vitamins, etc.) for maintenance or growth of NK cells. In an embodiment, culturing is performed in tissue culture dishes or flasks having the medium comprising the feeder layer and NK cell expansion media supplemented with IL-2. It refers to growing cells under conditions suitable for expansion of NK cells. The culturing, according to the present disclosure, is performed to obtain activated NK cells (i.e. NK cells in an activated or dividing state). The term “activated” or “dividing” state, used interchangeably herein, means that the NK cells are in a highly proliferative state or having an increased proliferating potential, especially as compared to the first or initial population of NK cells. In an example, the activated state is achieved by cytokine mediated activation of NK cell proliferation. Culturing may be performed for a desired period or to reach the desired confluency of cells. In an embodiment, the culturing and expansion is performed for a period in the range of 14 to 21 days.

The NK cells are allowed to grow on the feeder layer. In an embodiment, the culturing is performed for a period of 14 to 21 days, wherein the temperature is maintained at around 34° C. to 37° C. with 5% CO₂. The feeder layer is required to be checked frequently. Killing of MSC in the feeder layer indicates elimination or exhaustion and, thus, requires replacement with fresh feeder layer. In an embodiment, the NK cells are removed from the already eliminated feeder layer and reseeded into fresh feeder layer, preferably on day 3-5 of culturing.

The ratio of MSC feeder layer to natural killer cells is preferably in the range of 1:1 to 1:10. In an embodiment, the ratio of MSCs and NK cells is in the range of 1:1 to 1:10.

The medium for expansion or production of NK cells, according to the present disclosure, is an NK cell expansion medium capable of proliferating NK cells. Various NK expansion media are known in the art and may be used in embodiments herein. In an example, ExCellerate™ Human NK Cell Expansion Media is used as NK expansion media. The media may further include effective amounts of one or more cytokines. In an embodiment herein, the media comprises IL-2 in an amount of 200 IU-1000 IU.

The NK cell expansion medium may be replaced every 2-4 days. In an embodiment, the NK cell expansion media comprising IL-2 is replaced every 2 to 4 days. In an embodiment, expansion is performed for a period of 14-21 days.

The method further includes a step of expanding the NK cells by repeatedly replacing the medium along with the feeder layer. The term “repeatedly replacing”, as used herein, refers to replacement of eliminated feeder layers and exhausted medium. The used feeder layer and medium may be replaced with fresh feeder layer and medium, and the culturing is continued to obtain expanded NK cells. It is understood that the replacement of media may be performed as and when required or desired, infinitely. The feeder layer expresses membrane bound IL-21 which facilitates the expansion of NK cells. In an embodiment, the method involves expanding the NK cells by repeatedly replacing the medium and the feeder layer at least one time. In an embodiment, the method comprises expanding the NK cells by repeatedly replacing the medium and the feeder layer 2 to 5 times. In another embodiment, expanding comprises removing the NK cells from the eliminated feeder layer and reseeding the cells into fresh feeder layer, preferably on day 5 of culturing, initially and then 2 to 4 days till harvest. This step may be repeated 2 to 5 times, or more.

Proliferation of NK cells using the feeder layer results in feeder layer elimination. Regular observation would be necessary to ensure the level of feeder layer elimination. In an example, once the feeder layer is eliminated, the NK cells are collected and centrifuged, followed by further resuspension using fresh media. The NK cells are then transferred to a new culture dish with fresh feeder layer. Upon feeder layer elimination, the cell suspension is collected from the existing dish and post centrifugation, resuspended into fresh media. The suspension is then transferred to bigger dishes or flasks with fresh feeder layer. The expanded NK cells are propagated in appropriate dishes until they are ready for harvest on day 14-21. Upon achieving the desired expansion, NK cells may be cryopreserved using suitable proprietary cryomedia. In an embodiment, the cryopreserved NK cells are stored in liquid nitrogen tanks.

### Producing CAR-NK Cells

Further, embodiments herein also provide a method for producing NK cells, particularly CAR-NK cells. In an embodiment, the method comprises providing the NK cells; introducing a heterologous gene encoding a chimeric antigen receptor (CAR) to the NK cells, to obtain CAR-NK cells; and culturing the CAR-NK cells in the presence of the feeder layer. In an embodiment, the method comprises providing the NK cells; introducing a heterologous gene encoding a chimeric antigen receptor (CAR) to the NK cells, to obtain CAR-NK cells; and culturing the CAR-NK cells in the presence of the feeder layer.

In an embodiment, the method comprises providing the NK cells or expanded NK cells; introducing, by transfecting or transducing, the NK cells with a heterologous gene encoding a chimeric antigen receptor (CAR), to obtain CAR-NK cells; culturing the CAR-NK cells in a medium, wherein the medium comprises a feeder layer comprising a population of Mesenchymal stem cells (MSCs) having disrupted or deleted MHC class I gene and/or MHC class II gene and a heterologous gene for expressing IL-21, and an effective amount of IL-2; and expanding the CAR-NK cells by repeatedly replacing the feeder layer and culturing the CAR-NK cells, to obtain expanded CAR-NK cells.

In an embodiment, the method comprises: (i) obtaining NK cells or expanded NK cells from the method described herein; (ii) transfecting or transducing the expanded NK cells with a heterologous gene encoding a chimeric antigen receptor (CAR), to obtain CAR-NK cells and (iii) culturing the CAR-NK cells in a medium to obtain activated CAR-NK cells, wherein the medium comprises: (a) a feeder layer having a population of mesenchymal stem cells (MSCs) comprising disrupted or deleted MHC class I gene and/or MHC class II gene and a heterologous gene for expressing Interleukin-21 (IL-21) and (b) an effective amount of IL-2; and (iv) expanding the activated CAR-NK cells by repeatedly replacing the feeder layer and culturing the CAR-NK cells, to obtain expanded CAR-NK cells. Embodiments of the method for producing NK cells or CAR-NK cells comprises providing NK cells, preferably expanded by the method disclosed herein; and introducing the NK cells with a heterologous gene encoding a chimeric antigen receptor (CAR), to obtain CAR-NK cells. The CAR modification may be performed to obtain any suitable CAR-NK, as described previously herein. Accordingly, embodiments herein provide a method for producing CAR-NK cells. In an embodiment, the method for producing CAR-NK cells comprises: (a) providing an initial population of NK cells, (b) transfecting or transducing the NK cells with a heterologous gene encoding a chimeric antigen receptor (CAR), to obtain CAR-NK cells, wherein the heterologous gene is a gene encoding a chimeric antigen receptor (CAR) for binding to tumor cell antigen(s) selected from a group consisting of CD19, B-cell maturation antigen (BCMA), CD123, CD138, CD20, CD22, CD38, CD5, Immunoglobulin kappa (IgK) Chain, Lewis Y (LeY) antigen, NKG2D ligand, Receptor Tyrosine Kinase Like Orphan Receptor 1 (ROR1) and Wilms' tumor 1 (WT1), HLA-G, PDL-1 and a heterologous gene encoding a cytokine Interleukin-15 (IL-15); and (c) culturing the CAR-NK cells in a medium, wherein the medium comprises: a feeder layer having a population of Mesenchymal stem cells (MSCs) comprising disrupted or deleted MHC class I gene and/or MHC class II gene and a heterologous gene encoding IL-21.

In an embodiment, the method for producing CAR-NK cells further comprises expanding the CAR-NK cells or activated CAR-NK cells by repeatedly replacing the feeder layer and culturing the CAR-NK cells to obtain expanded CAR-NK cells. Embodiments of the method for producing CAR-NK cells comprise providing NK cells. The NK cells, as previously described herein, may be NK cells derived from cord blood or healthy donors. In an embodiment, the NK cells are expanded cells obtained by an expansion method as disclosed herein. The NK cells, in an embodiment, are obtained by negative selection comprising enriching of CD56+ CD3− NK cells in PBMC by depletion of CD3+ T cell from PBMCs using biotin-conjugated monoclonal antibodies against CD3. For example, from the PBMC population, CD56+ CD3− NK cells may be enriched through a negative selection method, mainly to deplete CD3+ T cell contamination, using biotin-conjugated monoclonal antibodies against antigens not expressed by NK cells. Using magnetic separation, unlabeled cells would be collected as the flow through, representing the enriched NK cell population. To validate the enrichment, the cells would be subjected to flow cytometry to determine the percentage of CD56 positive cells, where CD3 would be used as the negative control. The PBMC derived NK cells may then be transfected with one or more genes for immortalization of NK cells which may further be used in producing CAR-NK cells, in accordance with the embodiments herein.

Successfully engineered CAR-NK cells may be validated using a CD19 antibody against the CAR epitope. The inserted CAR transgene sequence may also be validated by qRT-PCR. Other systems such as lipofection or viral transduction may also alternatively be used. In an example, the plasmid for transfection is incubated with lipofectamine to form lipid droplets comprising the plasmid which can then pass through the cellular membrane to achieve the transfected cell.

Embodiments of the method for producing CAR-NK cells comprise culturing the CAR-NK in a medium. The culturing step is performed, as previously described herein, in a medium comprising a feeder layer comprising a population of Mesenchymal stem cells (MSCs) having disrupted or deleted MHC class I gene and/or MHC class II gene and a heterologous gene encoding IL-21; and an effective amount of IL-2. The CAR-NK, in an embodiment, expresses IL-15, which facilitates CAR-NK cell proliferation.

The method for producing CAR-NK cells comprises a step of expanding the activated CAR-NK by repeatedly replacing the medium along with the feeder layer. The eliminated feeder layer and medium may be replaced with fresh feeder layer and medium, and the culturing is continued to obtain CAR-NK. The IL-21 expressed by the feeder layer and IL-15 expressed by CAR-NK cells facilitate cell proliferation. Expansion of the activated CAR-NK cells is by repeatedly replacing the medium and the feeder layer at least one time. In an embodiment, the method comprises expanding the activated CAR-NK by repeatedly replacing the medium and the feeder layer 2 to 5 times.

In an example, once the feeder layer is eliminated, the CAR-NK cells are collected and centrifuged, followed by further resuspension using fresh media. The CAR-NK cells may then be transferred to a new culture dish with fresh feeder layer with 60-80% confluency. Upon feeder elimination, the cell suspension is collected from the existing dish and post centrifugation, resuspended into fresh media. The suspension is then transferred to a fresh feeder layer. Likewise, depending on the cell density, expanded CAR-NK cells are propagated in appropriate cell culture flasks until they are ready for harvest on day 14-21.

Upon achieving the desired expansion, the CAR-NK cells may be cryopreserved using suitable cryomedia.

The expanded population of NK or CAR NK cells, obtained by the method according to embodiments herein, may be used in various therapeutic applications, for e.g.: cancer treatment, anti-tumor applications, etc. Accordingly, in an embodiment, the present disclosure provides an expanded population of NK or CAR NK cells for use in treating a patient in need thereof. Patient, according to the present disclosure, may be a cancer patient having B-cell malignancies, B-cell acute lymphoblastic leukemia (B-ALL), chronic lymphocytic leukemia (CLL), and B-cell non-Hodgkin lymphoma (B-NHL), etc. The expanded population of NK or CAR NK cells may be administered to the patient using methods known in the art.

Further, embodiments herein provide a feeder layer for expansion of NK cells or CAR-NK cells. In an embodiment, the feeder layer comprises a population of mesenchymal stem cells (MSCs) having disrupted or deleted MHC class I gene and/or MHC class II gene and a heterologous gene for expressing Interleukin-21 (IL-21). In an embodiment, the feeder layer comprises MSCs that express membrane bound Interleukin-21 (IL-21).

Further, embodiments herein provide a medium for expansion of NK cells or CAR-NK cells, said medium comprising the feeder layer, wherein the feeder layer comprises a population of mesenchymal stem cells (MSCs) having disrupted or deleted MHC class I gene and/or MHC class II gene and a heterologous gene for expressing Interleukin-21 (IL-21); and an effective amount of Interleukin-2 (IL-2). The IL-2 is added extraneously to the medium. In an embodiment, the medium is an NK expansion medium comprising the population of mesenchymal stem cells (MSCs) having disrupted or deleted MHC class I gene and/or MHC class II gene and a heterologous gene for expressing Interleukin-21 (IL-21); and the Interleukin-2 (IL-2). In an embodiment, the effective amount of IL-2 is an amount in the range of 200 IU-1000 IU in respect of the medium.

### Method for Evaluation of CAR-NK Cells

Embodiments herein provide a method for evaluation of CAR-NK cells for cytotoxic activity. In an embodiment, the method for evaluating CAR-NK cells comprises providing the CAR-NK; evaluating the CAR-NK by co-culturing, in-vitro, the expanded CAR-NK and a tester cell line in a medium, wherein the tester cell line comprises induced Pluripotent stem cells (iPSCs) comprising disrupted or deleted MHC class I gene and/or MHC class II gene and a heterologous gene encoding a tumor antigen; and determining cytotoxicity by measuring cell death or cell damage in the tester cell line. The medium for co-culturing the expanded CAR-NK cells and a tester cell line in the evaluation method, according to embodiments herein, comprises NK cell expansion media described previously herein.

In another embodiment, the method for evaluating CAR-NK cells comprises, providing an animal comprising an implant of a tester line, wherein the tester line comprises induced Pluripotent stem cells (iPSCs) comprises disrupted or deleted MHC class I gene and/or MHC class II gene and a heterologous gene encoding a tumor antigen; introducing the expanded CAR-NK, preferably obtained by the methods as described herein, into the animal; and determining cytotoxicity, in vivo, by measuring cell death or cell damage in the tester cell line or the tumor cell mass. In an example, the iPSC is introduced to the animal to form a teratoma followed by infusion of different dosage of CAR-NK cells into the animal, to determine the efficacy of the therapy in terms of increase of animal's life expectancy and/or reduction in the tumor growth. The tester line may further include a gene for expressing luciferase to enable luciferin-luciferase facilitated bioluminescent imaging. The expression of luciferase in the tester cells and in turn in the teratoma may be used in quantification or measuring cell death or cell damage.

Accordingly, in an embodiment, the method for evaluating CAR-NK cells comprises, providing an animal model comprising an implant of a tester line or an animal comprising an implant of tumor, wherein the tester line comprises induced pluripotent stem cells (iPSCs) comprising disrupted or deleted MHC class I gene and/or MHC class II gene, a heterologous gene encoding a tumor antigen, and a gene for expressing luciferase; introducing the expanded CAR-NK, preferably obtained by the methods as described herein, into the animal model; and determining cytotoxicity, in vivo, by measuring cell death or cell damage in the tester cell line or the tumor cell mass.

### Cytotoxicity Assay

Embodiments of the method for evaluation, according to the present disclosure, comprises determining cytotoxicity by measuring cell death or cell damage in the tester cell line. Various methods for determining cell death and cell damage are known in the art, any of which may be used in various embodiments herein. Cell death may be determined by trypan blue staining or propidium iodide staining to identify the dead cells. Further, viability assays such as calcein AM staining, wherein the non-fluorescent calcein AM is converted to green fluorescent calcein, in live cell, after acetoxymethyl ester hydrolysis by intracellular esterase. Quantification of the fluorescence provides a measure of cell death or cell damage. Colorimetric assay-based detection of Glucose-6-Phosphate dehydrogenase or lactate dehydrogenase released from damaged cells may also be used in determining cell death and cell damage. In an embodiment, the cell death or cell damage is measured using a technique selected from trypan blue staining, propidium iodide staining, calcein AM staining, colorimetric assay, luciferase assay, or combination thereof. A vast array of assays for detecting cell viability are known and may be used in determining cytotoxicity, for e.g.: refer thermofisher.com/in/en/home/life-science/cell-analysis/cell-viability-and-regulation/cell-viability.html.

### Tester Line

Embodiments herein include a tester line. The term “tester line” or “tester cell line”, as used herein, refers to a cell line comprising modified induced pluripotent cells (iPSCs), wherein the iPSCs comprise a heterologous gene encoding an antigen for e.g.: tumor antigen. In an embodiment, the tester line is an iPSC cell line comprising a heterologous gene encoding a tumor antigen selected from CD19, B-cell maturation antigen (BCMA), CD123, CD138, CD20, CD22, CD38, CD5, Immunoglobulin kappa (IgK) Chain, Lewis Y (LeY) antigen, NKG2D ligand, Receptor Tyrosine Kinase Like Orphan Receptor 1 (ROR1) and Wilms' tumor 1 (WT1). In an embodiment, the tester cell line comprises iPSC cells comprising a heterologous gene comprising a nucleotide for expressing the tumor antigen, wherein the nucleotide has a sequence as set forth in SEQ ID NO: 5.

The tester cell line, according to embodiments herein, comprises modified induced pluripotent cells comprising a disrupted or deleted MHC class I and/or MHC class II genes. Accordingly, the iPSC tester cell line is incapable of expressing MHC class I and/or class II.

In an embodiment, the tester cell line comprises iPSCs comprising disrupted or deleted MHC class I genes and/or MHC class II genes. In an embodiment, the tester cell line comprises iPSCs having disrupted or deleted MHC class I genes and MHC class II genes by disrupting B2M and CIITA genes, respectively.

In an embodiment, the tester cell line comprises iPSCs comprising (a) disrupted or deleted MHC class I gene, wherein the MHC class I gene is B2M gene; and/or disrupted or deleted MHC class II gene, wherein the MHC class II gene is CIITA gene (b) a heterologous gene encoding a tumor antigen selected from CD19, B-cell maturation antigen (BCMA), CD123, CD138, CD20, CD22, CD38, CD5, Immunoglobulin kappa (IgK) Chain, Lewis Y (LeY) antigen, NKG2D ligand, Receptor Tyrosine Kinase Like Orphan Receptor 1 (ROR1) and Wilms' tumor 1 (WT1).

In another embodiment, the tester cell line comprises iPSCs comprising (a) disrupted or deleted MHC class I gene, wherein the MHC class I gene is B2M gene; and/or disrupted or deleted MHC class II gene, wherein the MHC class II gene is CIITA gene; and (b) a heterologous gene comprising a nucleotide for expressing CD19, wherein the nucleotide has a sequence as set forth in SEQ ID NO: 5.

The tester cell line, according to present disclosure, may be achieved using gene editing methods generally known in the art. The tester cell lines may be maintained in pluripotent stem cell maintenance media and passaged at 80-90% confluency with commercially available cell dissociation media suitable for the iPSC culture. In an embodiment, the tester lines are MHC class I and/or MHC class II null human iPSCs (hiPSC) expressing CD19, maintained in suitable media (for e.g.: mTeSR™ Plus, StemFlex, E8 Flex). To create a master cell bank, the iPSCs may be cryopreserved using cryomedia with 10% DMSO. The iPSC tester lines may go through several routine tests for quality control, including viability tests after thawing (every batch) using trypan blue staining, followed by counting of percentage viable cells.

The tester cell line, according to embodiments herein, is used to evaluate the cytotoxicity potential of the CAR-NK cells. The tester cell line, in an embodiment, may be maintained in an in-vitro culture system, wherein it enables the in-vitro evaluation of the CAR-NK cells. In another embodiment, the tester cell line may be implanted into an animal to form a teratoma, to obtain an animal model for evaluation of the CAR-NK cells in-vivo.

Accordingly, embodiments herein also provide an in-vitro/in-vivo model for evaluation of CAR-NK cells. In an embodiment, the model is a cell culture system comprising a tester line, wherein the tester line comprises iPSCs having disrupted or deleted MHC class I genes and/or MHC class II genes; and a heterologous gene encoding a tumor antigen selected from CD19, B-cell maturation antigen (BCMA), CD123, CD138, CD20, CD22, CD38, CD5, Immunoglobulin kappa (Igκ) Chain, Lewis Y (LeY) antigen, NKG2D ligand, Receptor Tyrosine Kinase Like Orphan Receptor 1 (ROR1) and Wilms' tumor 1 (WT1). The in-vitro system, in some embodiments, may be maintained at conditions that facilitate binding of the CAR-NK cells to the antigen on the iPSC tester line, preferably for a period of 2 to 24 hours.

In an embodiment, the co-culture of CAR-NK cells and tester cell line is maintained for about 24 h, wherein the CAR-NK cells and tester cell line is in a ratio of 1:1 to 1:30.

In another embodiment, the model is an animal model comprising an implant of the tester cell line, wherein the tester cell line comprises iPSCs having disrupted or deleted MHC class I genes and/or MHC class II genes; and a heterologous gene encoding a tumor antigen selected from CD19, B-cell maturation antigen (BCMA), CD123, CD138, CD20, CD22, CD38, CD5, Immunoglobulin kappa (IgK) Chain, Lewis Y (LeY) antigen, NKG2D ligand, Receptor Tyrosine Kinase Like Orphan Receptor 1 (ROR1) and Wilms' tumor 1 (WT1). The animal may suitably be implanted with the tester cell line, using techniques known in the art, to achieve the animal model. Introducing tumor cell transplants into immunocompromised mice for generating mice cancer models for drug testing is a known strategy. The tester line, according to embodiments herein, may be introduced into an immunocompromised mouse, using generally known transplantation techniques, to obtain a mouse model. Further, in some embodiments, the animal may further be induced to express luciferase enzyme enabling luciferin-luciferase facilitated bioluminescent imaging for sacrifice-free evaluation of cytotoxicity. In vivo bioluminescent imaging (BLI) is increasingly being utilized as a method for modern biological research, refer Dan M. Close, Tingting Xu, Gary S. Sayler, and Steven Ripp; In Vivo Bioluminescent Imaging (BLI): Noninvasive Visualization and Interrogation of Biological Processes in Living Animals; doi: 10.3390/s110100180.

### Gene Editing Methods

Gene editing in cells, in the present disclosure, may be performed using methods and tools generally known in the art. Examples of such methods and tools for gene editing include, but are not limited, transfection, electroporation, nucleofection, transduction using retroviral vectors, lentiviral vectors, transposons mediated gene editing, designer nucleases including zinc finger nucleases (ZFNs), transcription activator-like (TAL) effector nuclease or CRISPR/Cas (Clustered Regularly Interspaced Short Palindromic Repeats Associated 9) assisted gene editing, etc.

The disruption or deletion of MHC class I and class II gene, in various embodiments herein, may be performed by CRISPR/Cas gene editing system. Typically, the CRISPR/Cas system employs RNA-guided nucleases for gene editing. It uses Cas9 (a DNA nuclease) for strand specific cleavage, and two noncoding RNAs viz. crispr RNA (crRNA)—having a sequence complementary to a target DNA (or target sequence) and trans-activating RNA (tracrRNA)—an auxiliary RNA, in cleavage of the target DNA. The tracrRNA facilitates by hybridizing with the 3′ end of crRNA to form an RNA-duplex structure that is bound by the Cas9 nuclease to form the catalytically active CRISPR-Cas complex, which can then cleave the target DNA. Once the target DNA is cleaved the cell employs DNA repair mechanisms, for e.g.: non-homologous end-joining (NHEJ) and homology-directed repair (HDR) for sealing the DNA breaks produced by the CRISPR-Cas complex. Embodiments herein may employ the Type-I, Type-II or Type-II CRISPR-Cas system, or any CRISPR-Cas systems known in the art (Koonin et al., (2017) Curr Opin Microbiol 37:67-78).

Transfection, according to embodiments herein, is preferably performed by electroporation. In an embodiment, electroporation is by neon electroporation system. In an embodiment, transfection of heterologous gene encoding CAR is performed by electroporation. In an embodiment, the SV40LT and hTERT genes are introduced into cells by electroporation. It is understood that, in light of the present disclosures, various other transfection systems would be apparent to a person skilled in the art, which are all included within the scope of the present disclosures. Virus-mediated transduction is an example of another way to introduce the heterologous gene encoding the CAR.

Gene editing, in some embodiments, is performed by transposon-mediated gene editing, wherein two plasmids are co-transfected, wherein one plasmid for expressing anti CD19-CAR, flanked by 5′ and 3′ ITR in the transposon plasmid, the other plasmid for constitutively expressing the transposase under the control of a CAG promoter. In an embodiment, transfection of heterologous gene encoding CAR genes is performed using the transposon-mediated gene editing. Transduction with viruses is another way of introducing CAR genes.

Although the subject matter has been described with reference to specific embodiments, this description is not meant to be construed in a limiting sense. Various modifications of the disclosed embodiments, as well as alternative embodiments of the subject matter, will become apparent to persons skilled in the art upon reference to the description of the subject matter. It is therefore contemplated that such modifications can be made without departing from the spirit or scope of the present subject matter as defined.

### LISTING OF EMBODIMENTS

The subject matter disclosed herein includes, but is not limited to, the following embodiments.
1. A method for expansion of natural killer (NK) cells, comprising: (i) obtaining a first population of NK cells; (ii) culturing the NK cells in a medium to obtain activated NK cells, wherein the medium comprises: (a) a feeder layer having a population of mesenchymal stem cells (MSCs) comprising disrupted or deleted MHC class I gene and/or MHC class II gene and a heterologous gene for expressing Interleukin-21 (IL-21), and (b) Interleukin-2 (IL-2); and (iii) expanding the activated NK cells by repeatedly replacing the feeder layer, to obtain expanded NK cells.
2. The method of embodiment 1, wherein the first population of NK cells are chimeric antigen receptor-natural killer (CAR-NK) cells.
3. The method of embodiment 1, wherein the first population of NK cells is derived from peripheral blood mononuclear cells (PBMCs) or Umbilical cord blood (UCB), preferably by subjecting PBMCs to Magnetic activated cell sorting (MACS).
4. The method of embodiment 1, wherein said culturing in step (ii) is performed for a period of 2 to 5 days.
5. The method of embodiment 1, wherein said replacing the feeder layer in step (iii) is performed every 2-5 days, preferably for a period of 14-21 days.
6. A method for producing CAR-NK cells, comprising: (i) providing NK cells, or the expanded NK cells obtained by the method of embodiment 1; (ii) introducing a heterologous gene encoding a chimeric antigen receptor (CAR) into the NK cells or the expanded NK cells, to obtain CAR-NK cells; (iii) culturing the CAR-NK cells in a medium to obtain activated CAR-NK cells, wherein the medium comprises: (a) feeder layer having a population of Mesenchymal stem cells (MSCs) comprising disrupted or deleted MHC class I gene and/or MHC class II gene and a heterologous gene for expressing IL-21, and (b) Interleukin-2 (IL-2); and (iv) expanding the activated CAR-NK cells by repeatedly replacing the feeder layer, to obtain expanded CAR-NK cells.
7. The method of embodiment 1 or 6, wherein the media comprises IL-2 in an amount of 200 IU-1000 IU.
8. The method of embodiment 6, wherein said culturing in step (iii) is performed for a period of 2 to 5 days.
9. The method of embodiment 6, wherein said replacing the feeder layer in step (iv) is performed every 2-5 days, preferably for a period of 14-21 days.
10. The method of embodiment 1 or 6, wherein the MSCs are conditionally immortalized.
11. The method of embodiment 10, wherein the MSCs comprise an exogenous gene selected from a group consisting of: a gene encoding a protein selected from SV40 large T antigen (SV40LT), human telomerase reverse transcriptase (hTERT), or combination thereof; and a gene having a nucleotide sequence selected from SEQ ID NO: 3, SEQ ID NO: 4, or combination thereof.
12. The method of embodiment 1 or 6, wherein the MSCs are immortalized MSCs obtained by a process selected from: (a) providing a population of induced Pluripotent stem cells (iPSCs) comprising disrupted or deleted MHC class I gene and/or MHC class II gene and a heterologous gene for expressing IL-21; introducing a heterologous gene to the iPSCs for conditionally immortalizing the iPSCs; and culturing the iPSCs in a differentiating medium to obtain the MSCs; or (b) providing a population of induced Pluripotent stem cells (iPSCs) having disrupted or deleted MHC class I gene and/or MHC class II gene and a heterologous gene for expressing IL-21; culturing the iPSCs in a differentiating medium to obtain the MSCs; and introducing a heterologous gene to the MSCs for conditionally immortalizing the MSCs.
13. The method of any one of embodiments 1-12, wherein the MHC class I gene is B2M gene and the MHC class II gene is CIITA gene.
14. The method of any one of embodiments 1-12, wherein the heterologous gene for expressing IL-21 comprises a nucleotide having a sequence as set forth in SEQ ID NO: 2.
15. A method for evaluating CAR-NK cells, said method comprising: (a) providing CAR-NK cells or the expanded CAR-NK cells obtained by a method of embodiment 6; (b) co-culturing, in-vitro, the expanded CAR-NK cells, and a tester cell line in a medium, wherein the tester cell line comprises induced Pluripotent stem cells (iPSCs) comprising disrupted or deleted MHC class I gene and/or MHC class II gene and a heterologous gene encoding a tumor antigen; and (c) determining cytotoxicity by measuring cell death or cell damage in the tester cell line.
16. A method for evaluating natural killer (NK) cells, said method comprising: (a) providing an animal comprising an implant of a tester cell line or an animal comprising an implant of tumor, wherein the tester cell line comprises induced Pluripotent stem cells (iPSCs) comprising disrupted or deleted MHC class I gene and/or MHC class II gene and a heterologous gene encoding a tumor antigen; (b) introducing the NK cells or the expanded NK cells obtained by a method of embodiment 1, into said animal; and (c) determining cytotoxicity, in vivo, by measuring cell death or cell damage in the tester cell line or the implant.
17. The method of embodiment 15 or 16, wherein the tumor antigen is selected from a group consisting of CD19, B-cell maturation antigen (BCMA), CD123, CD138, CD20, CD22, CD38, CD5, Immunoglobulin kappa (IgK) Chain, Lewis Y (LeY) antigen, NKG2D ligand, Receptor Tyrosine Kinase Like Orphan Receptor 1 (ROR1) and Wilms' tumor 1 (WT1), PDL1 and HLA-G.
18. A model for evaluating CAR-NK cells, said model comprising a tester line, wherein the tester line comprises induced Pluripotent stem cells (iPSCs) comprising disrupted or deleted MHC class I gene and/or MHC class II gene and a heterologous gene for expressing a tumor antigen.
19. The method of any one of embodiments 15-18, wherein the tumor antigen is CD19 and wherein the heterologous gene comprises a nucleotide having a sequence as set forth in SEQ ID NO: 5.
20. An expanded population of NK or CAR NK cells obtained by a method of any one of embodiments 1-14, for use in treating a patient in need thereof.
21. A feeder layer for expansion of NK cells or CAR-NK cells, said feeder layer comprising a population of mesenchymal stem cells (MSCs) having disrupted or deleted MHC class I gene and/or MHC class II gene and a heterologous gene for expressing Interleukin-21 (IL-21).
22. A medium for expansion of NK cells or CAR-NK cells, said medium comprising the feeder layer of embodiment 21; and an effective amount of Interleukin-2 (IL-2).
23. The medium of embodiment 22, wherein the effective amount is an amount in the range of 200 IU-1000 IU in respect of the medium.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. All the media are prepared following the manufacturer's protocol. Although methods and materials similar, or equivalent to those described herein can be used in the practice of the disclosed methods and compositions, the exemplary methods, devices, and materials are described herein. It is to be understood that this disclosure is not limited to particular methods, and experimental conditions described, as such methods and conditions may vary.

### Example 1: Preparation of MSC Feeder Layer for Expansion of NK Cells

Human iPSCs carrying knockout (KO) mutations for 2 key components β2 Microglobulin (B2M) and class II major histocompatibility class transactivator (CIITA) of major histocompatibility complexes I and II (i.e., Human leukocyte antigen [HLA] I/II knockout hiPSCs) were generated using the CRISPR-Cas under standard electroporation conditions. The cells were expanded clonally, selected for homozygous KO, validated, and further expanded. MSCs were derived from these iPSCs using standardized protocols and iPSC derived MSCs were used as the starting material for immortalization.

### Example 2: MSC Immortalization Protocol Using Neon Electroporation System for Producing the Feeder Layer for Expansion of NK Cells

Non-Immortalized MSCs (refer Example 1) were dissociated using cell dissociation media. The cell suspension was centrifuged, and the pellet was resuspended in DPBS. The cells were counted using a haemocytometer. Thereafter, the cells were centrifuged again at 300 g for 3 minutes and the pellet was resuspended with R buffer, provided with the Neon Kit. A dish with MSC expansion media was prepared and kept in the incubator for conditioning. Plasmid with the genes encoding hTERT and SV40LT, and the associated transposase plasmid were diluted to achieve optimal concentration using buffer R provided with the Neon Kit. The plasmids were transferred to 1.5 ml Eppendorf tube along with the cell suspension, such that the final volume of the DNA-cell mixture was 30 μL. The electroporation was performed according to the manufacturer's protocol. Post electroporation, the cells were incubated at 37° C. with 5% CO₂. On the following day, the media was changed with MSC expansion media having 1 μg/mL doxycycline. After 72 hours, 100-200 μg/mL of Ncomycin G-418 was added to the dish. Any other selection antibiotic can also be used for this step. The antibiotic selection was done until all the untransfected control cells were dead. The selected cells were expanded for the next step.

### Example 3: Overexpression of IL-21 in the Immortalized MSC Feeder

As a further modification, the immortalized MSCs (refer Example 2) were electroporated with a transposon-based vector expressing membrane bound human IL-21 and suitable transposase expression vector. They were further selected with puromycin and expanded to be used as a feeder for NK cell expansion.

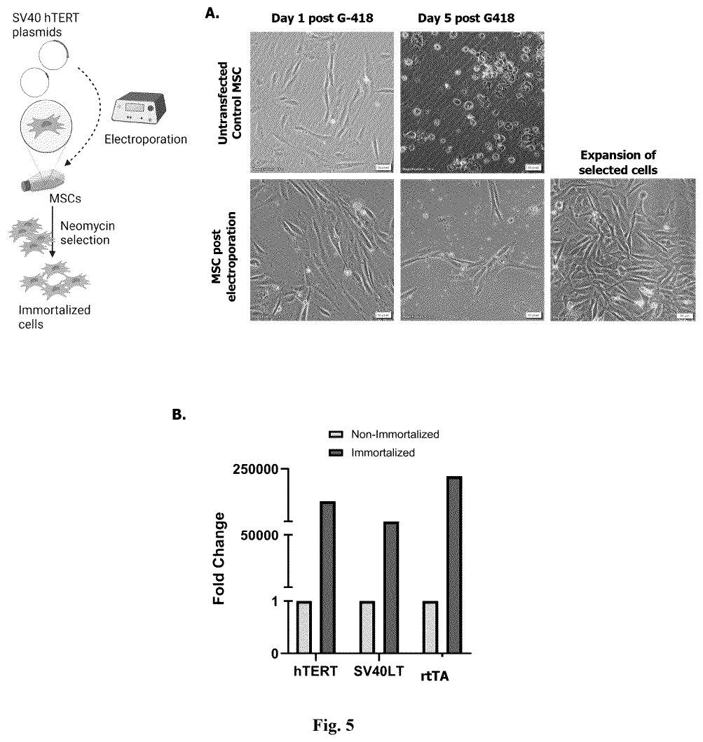

FIG. 5 shows MHC-I and II null MSC transfected with hTERT SV40 T-antigen plasmid to achieve immortalization. A. (Top panel) Untransfected control MSCs post neomycin selection at DAY 1 and DAY 5. (Bottom panel) Transfected MSCs post ncomycin selection at DAY 1 and DAY 5, followed by expansion of the selected cells. B. qPCR result showing differential expression of hTERT, SV40LT and rtTA compared to control cells.

### Example 4: Maintenance of Feeder Layer for the Expansion of NK Cells

Thawing of immortalized MSC feeder line: A vial of IL-21 MSC hTERT SV40 was taken from liquid nitrogen storage and gently hand-thawed and contents were transferred into a 15 mL falcon already containing 2.5 mL of pre-warmed complete MSC expansion media. The cell suspension was centrifuged for 3 minutes at 300 g at room temperature. The supernatant was discarded, and the pellet was resuspended in a complete culture medium with doxycycline. Post cell counting, if the cells presented a viability of lower than 70%, they were considered to be unsuitable for cell culture. Post plating in optimal density, the cells were maintained at 37° C.-5% CO₂ in a humidified incubator.

Maintenance of MSC feeder line: In order to maintain the MSC feeder line, the media was changed with complete MSC expansion media with 1 μg/mL of doxycycline the day after thawing. The culture medium was changed every 2-3 days with complete MSC expansion media with 1 μg/mL of doxycycline. The cells were stored until ˜80% confluent, after which they were harvested.

Harvesting of MSC feeder layer: Before harvesting, appropriate culture ware was coated with 5% human AB serum. The existing culture media in the dish was discarded and the cell monolayer was washed with 1×DPBS. Cell dissociation media was added, and the cells were incubated at 37° C. for 3-4 minutes. The action of cell dissociation media was blocked by adding the same volume of fresh complete pre-warmed media as the volume of cell dissociation media. The cell suspension was transferred into sterile 15 mL centrifugation tubes and centrifuged for 3 minutes at 300 g. After centrifugation, the supernatant was discarded, and the pellet was resuspended in a complete culture medium with 1 μg/mL doxycycline. The cells were plated as per the required split ratio (usually 1:3) or based on cell density depending on requirements and were incubated at 37° C.-5% CO₂ in a humidified incubator.

Freezing of MSC feeder layer: The existing culture media in the dish was discarded and the cell monolayer was washed with 1×DPBS. Cell dissociation media was added, and the dish was incubated at 37° C. for 3-4 minutes. The action of cell dissociation media was blocked by adding the same volume of fresh complete pre-warmed media as the volume of cell dissociation media. The cell suspension was transferred into sterile 15 mL centrifugation tubes and centrifuged for 3 minutes at 300 g. After centrifugation, the supernatant was discarded, and the pellet was gently resuspended in ice cold Cryomedia with 10% DMSO to reach the desired cell concentration (the desired cell concentration was usually 10⁶ cells/0.5 mL of cryomedia with 10% DMSO). The cell suspension was transferred to a −80° C. freezer followed by a liquid nitrogen tank for long-term storage.

Quality control of the MSC cells: For checking the quality of the MSC cells, multiple procedures were followed. The freeze-thaw viability of the cells was checked for >70% viability. After 10 passages, standard quality control assays like karyotyping (cytogenetic stability), gene expression and immunophenotyping, and sterility testing were carried out for every MSC line. If required, flow cytometry for cell characterization was also done with CD105 and CD90 (positive markers). The expression of membrane bound IL-21 was checked by qPCR.

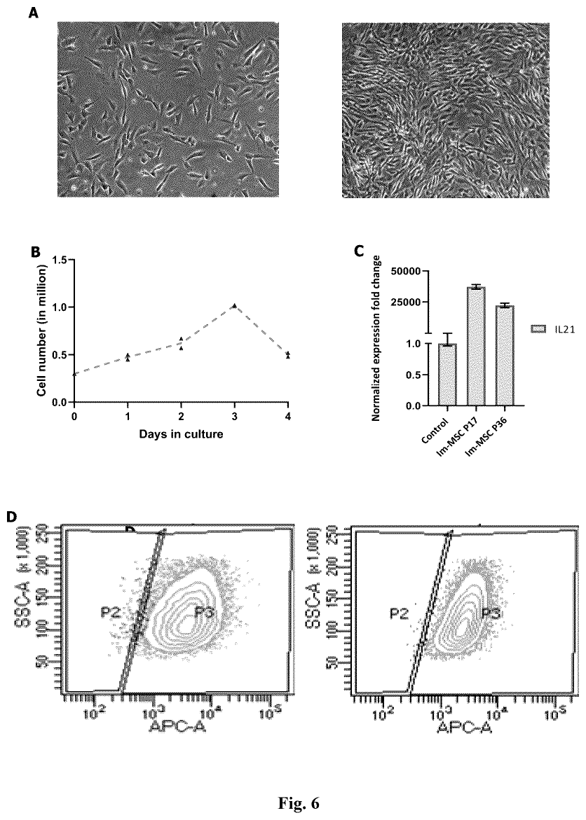

FIG. 6, panel A, illustrates representative images showing Mesenchymal Stem cell (MSC) feeder morphology in culture at 24 hours (top) and at 48 hours (bottom) B. Line plot showing the growth pattern of the feeder cells. The population doubling time is ˜ 40 hours. 2 data points representing 2 experimental repeats. C. Bar graphs showing the expression fold change of membrane bound IL-21 between control and membrane bound IL-21 overexpressing feeder cells at early (P17) and late passages (P36). D. Contour plot showing the flow cytometry results characterizing the MSC cells by expressing CD90 and CD105.

### Example 6: Expansion of NK Cells by NK-MSC Feeder Layer Co-Culture

Isolate PBMC from buffy coat: One unit of the leukapheresis product was subjected to density gradient centrifugation using Ficoll-Paque. The buffy-coat layer was collected and washed multiple times with DPBS to eliminate Ficoll. The cell suspension was centrifuged to get PBMCs as cell pellet, which is further resuspended into proprietary cryomedia and stored in liquid nitrogen.

Enrichment of NK cells using magnetic sorting: 5×10⁷ to 10×10⁷ PBMCs were subjected to magnetic bead based negative selection using manufacturer's protocol. Enriched NK cells were plated with hTERT SV40 IL-21 overexpressing immortalized MSC feeder cells.

Maintenance of NK cells: After 2-5 days of culture PBMC-NK cells were replated on fresh MSC feeder using fresh NK expansion media with IL-2, after centrifuging the cells in 200 g for 7 mins. Feeder killing was observed under microscope before replating. Every 2-5 days cells were counted to understand the expansion fold change.

Freezing of NK.pure cells: Upon culturing for 14-21 days using hTERT SV40 IL-21 overexpressing MSC feeder cells, expanded NK cells were collected, centrifuged at 200 g for 7 minutes. After discarding the supernatant, the cell pellet was resuspended in proprietary cryomedia and stored in liquid nitrogen.

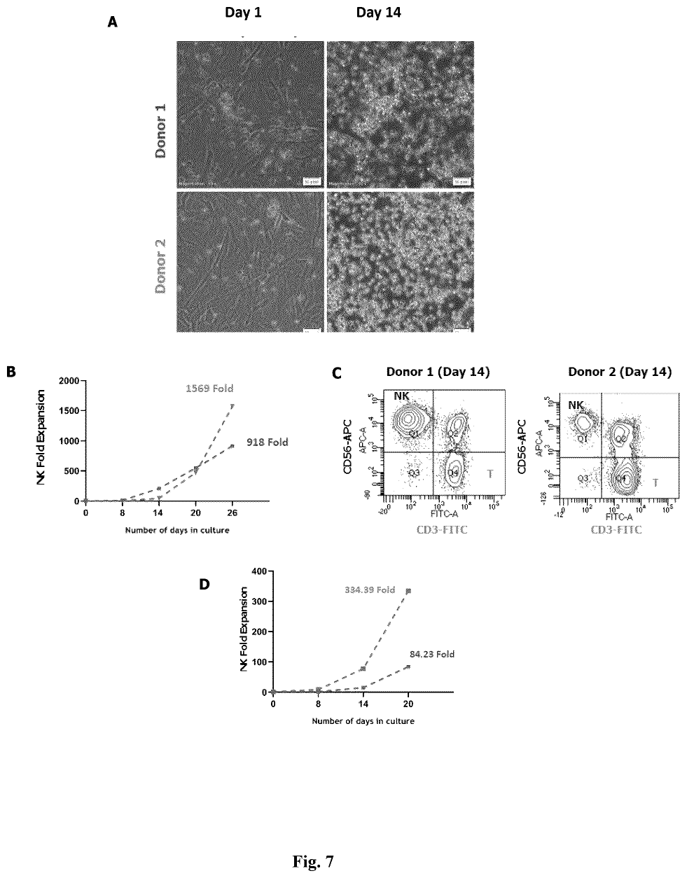

Quality Control of NK cells: To make sure that the NK cells retain their phenotype, flow cytometry was performed with CD56 Monoclonal antibody as a positive marker and CD3 Monoclonal antibody as a negative marker. Various other assays could also be carried out based on requirements, such as cytokine activation assays or metabolic activity measuring assays. FIG. 7 depicts the results for NK cells isolated from PBMCs expanded with proprietary media on the immortalized mbIL-21 overexpressing feeder with IL-2. The images in FIG. 7, panel A, show the expansion of PBMC population at Day 1 and Day 14 with feeder cell line, in accordance with the embodiments herein. FIG. 7, panel B, are line plots showing fold expansion of NK cells using the feeder layer and method, in different PBMC samples from two donors according to the embodiments herein, at Day 23; FIG. 7, panel C, is a contour plot depicting purity of NK cells (Q1) after 14 days of expansion, FIG. 7, panel D, are line plots showing attenuated fold expansion of NK cells without feeders in different PBMC samples from two donors, in accordance with the embodiments herein.

### Example 7: Preparing CAR-NK Cells

The expanded NK cells (refer Example 6) are subjected to electroporation or viral mediated transduction using Adeno-associated virus or lentivirus. For example, 3-4×10⁶ NK cells are used per condition to transfect or transduce plasmid or viral particles with FMC63 CAR against CD19. Before and after gene delivery, NK cells were maintained without feeder only using NK expansion media along with IL-2 until cells are selected. After maintaining the cells for at least 2 passages, NK cells were sorted using a monoclonal antibody against FMC63 CAR. Sorted cells are maintained with MSC feeder cells for further expansion. Expanded NK cells are cryopreserved using proprietary media in liquid nitrogen storage.

### Example 8: Evaluating CAR-NK Cells

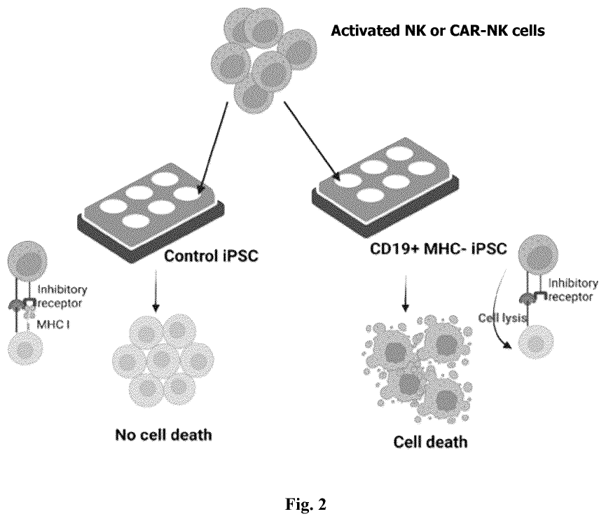

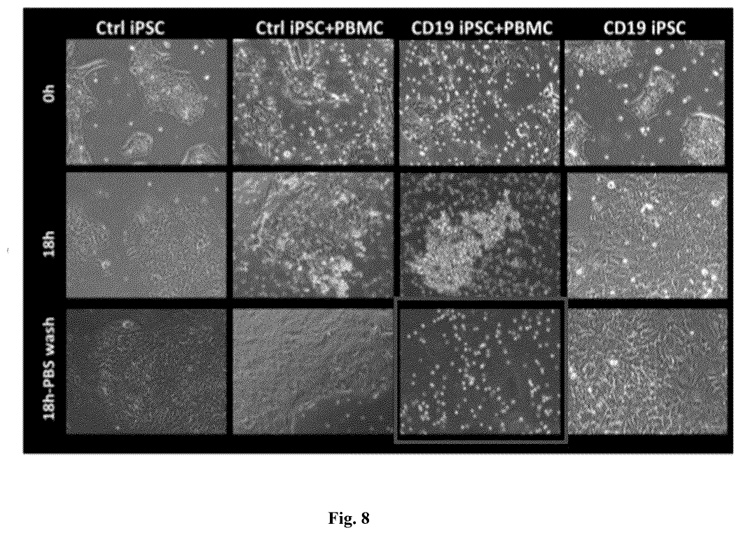

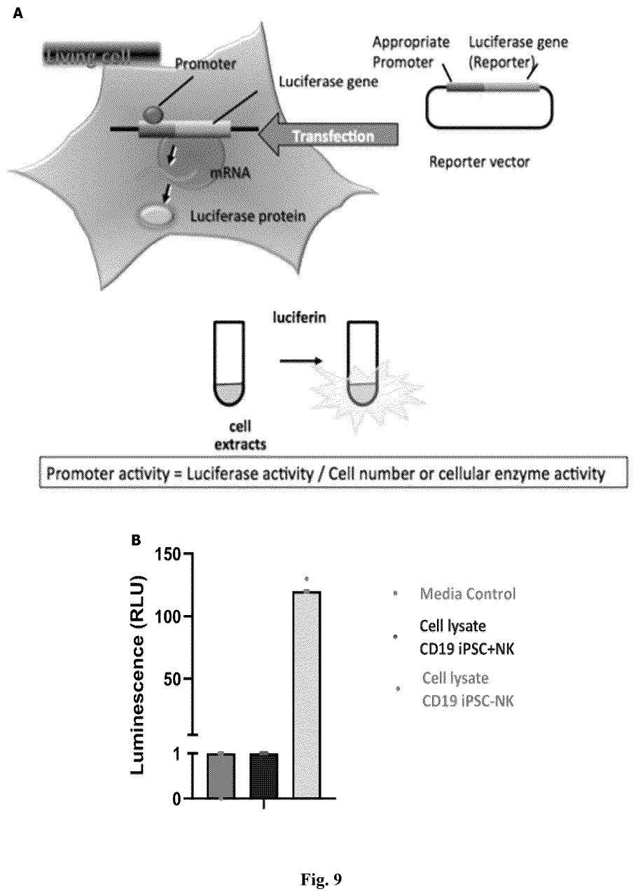

To evaluate the cytotoxicity of NK/CAR-NK cells, the tester iPSC cells (MHC class I and/or MHC class II null) expressing specific cancer related markers like CD19 cultured for 1-2 days in the iPSC specific proprietary media. After iPSC colonies have been established, NK/CAR-NK cells were added in the tester iPSC culture in 1:1 to 1:10 ratios. The media was then changed to NK media. FIG. 8 refers to images depicting target specific cytotoxicity results of expanded NK cells using the CD19 iPSC tester cell line. A similar experiment was also done with the control iPSC with intact MHC I and MHC II gene using the same NK: iPSC ratio to evaluate the target specificity of the NK/CAR-NK cells. A visual cytotoxicity score was assigned according to a 5-point rating scale, ranging from 1=cytotoxic to 5=non-cytotoxic, based on observable characteristics of cell proliferation and cell lysis. Alternatively, a luciferase assay system such as ONE-Glo™ Luciferase Assay System by Promega can be used (ONE-Glo™ Luciferase Assay System). FIG. 9, panel A, is a schematic representation depicting luciferase assay system, and FIG. 9, panel B, shows luminescence results, in accordance with the embodiments herein. The luciferase activity was detected as per manufacturer's instructions. FIG. 2 is a schematic representation depicting the method for evaluation of CAR-NK cells.

### Advantages of the Present Disclosure

The present disclosure provides a method for manifold expansion of natural killer cells. The present method is capable of achieving about 100 to 1000 or about 900 to 1000 expansion of NK cells, in about 14-21 days. Further, the present invention also achieves a method for production of CAR-NK cells. The present method employs MHC Class-I and/or Class-II null MSC feeder layer, wherein the MSCs are capable of expressing IL-21 in achieving NK cells, CAR-NK cells, using an NK cell expansion media comprising IL-2. The feeder layer of the present disclosure may be conditionally immortalized, thus enabling MSCs with a faster population doubling time, being a continuous source of the NK cell expansion platform for long term use. Further, the iPSC tester line, in accordance with the present disclosure, provides convenient and precise evaluation of CAR-NK cytotoxicity for immunotherapy.

I/We claim:

1. A method for expansion of natural killer (NK) cells, comprising:
(i) obtaining a first population of NK cells;
(ii) culturing the NK cells in a medium to obtain activated NK cells, wherein the medium comprises:
(a) a feeder layer having a population of mesenchymal stem cells (MSCs) comprising disrupted or deleted MHC class I gene and/or MHC class II gene and a heterologous gene for expressing interleukin-21 (IL-21), and
(b) interleukin-2 (IL-2); and
(iii) expanding the activated NK cells by repeatedly replacing the feeder layer, to obtain expanded NK cells.

2. The method of claim 1, wherein the first population of NK cells are chimeric antigen receptor-natural killer (CAR-NK) cells.

3. The method of claim 1, wherein the first population of NK cells is derived from peripheral blood mononuclear cells (PBMCs) or umbilical cord blood (UCB), preferably by subjecting PBMCs to magnetic activated cell sorting (MACS).

4. The method of claim 1, wherein said culturing in step (ii) is performed for a period of 2 to 5 days.

5. The method of claim 1, wherein said replacing the feeder layer in step (iii) is performed every 2-5 days, preferably for a period of 14-21 days.

6. A method for producing chimeric antigen receptor-natural killer (CAR-NK) cells, comprising:
(i) providing NK cells, or the expanded NK cells obtained by the method of claim 1;
(ii) introducing a heterologous gene encoding a chimeric antigen receptor (CAR) into the NK cells or the expanded NK cells, to obtain CAR-NK cells;
(iii) culturing the CAR-NK cells in a medium to obtain activated CAR-NK cells, wherein the medium comprises:
(a) feeder layer having a population of mesenchymal stem cells (MSCs) comprising disrupted or deleted MHC class I gene and/or MHC class II gene and a heterologous gene for expressing interleukin-21 (IL-21), and
(b) interleukin-2 (IL-2); and
(iv) expanding the activated CAR-NK cells by repeatedly replacing the feeder layer, to obtain expanded CAR-NK cells.

7. The method of claim 1, wherein the media comprises IL-2 in an amount of 200 IU-1000 IU.

8. The method of claim 6, wherein said culturing in step (iii) is performed for a period of 2 to 5 days.

9. The method of claim 6, wherein said replacing the feeder layer in step (iv) is performed every 2-5 days, preferably for a period of 14-21 days.

10. The method of claim 1, wherein the MSCs are conditionally immortalized.

11. The method of claim 10, wherein the MSCs comprise an exogenous gene selected from a group consisting of:
a gene encoding a protein selected from SV40 large T antigen (SV40LT), human telomerase reverse transcriptase (hTERT), or combination thereof; and
a gene having a nucleotide sequence selected from SEQ ID NO: 3, SEQ ID NO: 4, or combination thereof.

12. The method of claim 1, wherein the MSCs are immortalized MSCs obtained by a process selected from:
(a) providing a population of induced pluripotent stem cells (iPSCs) comprising disrupted or deleted MHC class I gene and/or MHC class II gene and a heterologous gene for expressing IL-21; introducing a heterologous gene to the iPSCs for conditionally immortalizing the iPSCs; and culturing the iPSCs in a differentiating medium to obtain the MSCs; or
(b) providing a population of induced pluripotent stem cells (iPSCs) having disrupted or deleted MHC class I gene and/or MHC class II gene and a heterologous gene for expressing IL-21; culturing the iPSCs in a differentiating medium to obtain the MSCs; and introducing a heterologous gene to the MSCs for conditionally immortalizing the MSCs.

13. The method of claim 1, wherein the MHC class I gene is B2M gene and the MHC class II gene is CIITA gene.

14. The method of claim 1, wherein the heterologous gene for expressing IL-21 comprises a nucleotide having a sequence as set forth in SEQ ID NO: 2.

15. A method for evaluating chimeric antigen receptor-natural killer (CAR-NK) cells, said method comprising:
(a) providing CAR-NK cells or the expanded CAR-NK cells obtained by the method of claim 6;
(b) co-culturing, in-vitro, the expanded CAR-NK cells, and a tester cell line in a medium, wherein the tester cell line comprises induced pluripotent stem cells (iPSCs) comprising disrupted or deleted MHC class I gene and/or MHC class II gene and a heterologous gene encoding a tumor antigen; and
(c) determining cytotoxicity by measuring cell death or cell damage in the tester cell line.

16. A method for evaluating natural killer (NK) cells, said method comprising:
(a) providing an animal comprising an implant of a tester cell line or an animal comprising an implant of tumor, wherein the tester cell line comprises induced pluripotent stem cells (iPSCs) comprising disrupted or deleted MHC class I gene and/or MHC class II gene and a heterologous gene encoding a tumor antigen;
(b) introducing the NK cells or the expanded NK cells obtained by the method of claim 1, into said animal; and
(c) determining cytotoxicity, in vivo, by measuring cell death or cell damage in the tester cell line or the implant.

17. The method of claim 15, wherein the tumor antigen is selected from a group consisting of CD19, B-cell maturation antigen (BCMA), CD123, CD138, CD20, CD22, CD38, CD5, immunoglobulin kappa (IgK) chain, Lewis Y (LeY) antigen, NKG2D ligand, receptor tyrosine kinase like orphan receptor 1 (ROR1) and Wilms' tumor 1 (WT1), PDL1 and HLA-G.

18. A model for evaluating chimeric antigen receptor-natural killer (CAR-NK) cells, said model comprising a tester line, wherein the tester line comprises induced pluripotent stem cells (iPSCs) comprising disrupted or deleted MHC class I gene and/or MHC class II gene and a heterologous gene for expressing a tumor antigen.

19. The method of claim 15, wherein the tumor antigen is CD19 and wherein the heterologous gene comprises a nucleotide having a sequence as set forth in SEQ ID NO: 5.

20. An expanded population of natural killer (NK) or chimeric antigen receptor-natural killer (CAR-NK) cells obtained by the method of claim 1, for use in treating a patient in need thereof.

21. A feeder layer for expansion of natural killer (NK) cells or chimeric antigen receptor-natural killer (CAR-NK) cells, said feeder layer comprising a population of mesenchymal stem cells (MSCs) having disrupted or deleted MHC class I gene and/or MHC class II gene and a heterologous gene for expressing interleukin-21 (IL-21).

22. A medium for expansion of natural killer (NK) cells or chimeric antigen receptor-natural killer (CAR-NK) cells, said medium comprising the feeder layer of claim 21; and an effective amount of interleukin-2 (IL-2).

23. The medium of claim 22, wherein the effective amount is an amount in the range of 200 IU-1000 IU in respect of the medium.


SEQUENCE LISTING (5 sequences)

SEQ ID NO: 1 - DNA, 399 bp, synthetic construct
  source 1..399: mol_type = other DNA; note = Nucleotide sequence encoding IL-15
ggcatacatgtcttcatattgggctgctttagcgcgggacttccaaagactgaggctaactgggtcaatgtgatcagcgacttgaagaagatcgaagacctgattcagtctatgcacatagacgccactctttacacggagtctgatgtgcatcccagttgtaaagtaacggcgatgaagtgctttcttcttgaattgcaagtgatctctcttgaatcaggggatgcaagtatacatgacaccgtagagaacctgataatactcgctaataactcattgtcctccaacggaaatgtgacggagagtggctgcaaggaatgcgaggaactggaagagaagaacatcaaggaatttcttcaaagtttcgttcatattgtgcagatgttcatcaacacaagt

SEQ ID NO: 2 - RNA, 3146 nt, Homo sp.
  source 1..3146: mol_type = other RNA; note = Nucleotide sequence encoding IL21
ctcagtcaagctgaagtgaaaacgagaccaaggtctagctctactgttggtacttatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctgaggatctaacttgcagttggacactatgttacatactctaatatagtagtgaaagtcatttctttgtattccaagtggaggagtacaatatattagcgatgggaaaaaaaaactcataagtgtgcaaagtcaggattatttccccataatcactatacaatagtctgatttcctatgttattgatttctatgacttctgaagtttttactttatctcactgcccaactttgacatttcttggttgaaggagaaggctaaaatttttaaccaaaaatacagttttgacatctattgattttatattaaccttgagcttgattaccaccagaatgaggtctattatgatcataacttaaaacatatataccttcattccagctgataatatagaagcatagaagcaaaatatataaaagcaaaatgaaaagttgacatctttattcttttaaggaaatgaaaactgagtgtttaaaaacttatatctgaatagttttttaaatctcagaatgtctaacatataaatatgtaatttatttatgcaccgggggatactagatggctataaaagccttacttctttattggggtttataaaaatatttaaatactatagatatgaatgtatttatggaatagactcttagtcagtggcaatagagtattaagaagtggatttaatattaccttcctccagcccccagtccccttataagctaacattttaagcttgtggaaatggcaatttaaacctatagagggacatatgccaggttgtcttctggaaccaaacatagttttaaaagtattgtagctctcttctcaaactgttataatgatcatattatgagcaagctagtaactccaaatatttgtctccttctttcttctttcgtttcttttcttccttctttttaaaatgaaacttctgctttgacaaaatttcacaacatgtttaacattttggacacttatagttactatactttggataagacaaattcaatcacctatataatttaaaaatcaggagacaaaccaagaggccaggactcctgtccaagttgaaaacagactttcgcataagcattccaatctttcagacttgggtagtaaaaacaagcaacctactctacctgccatttcatatcaactccagcacagcttgtttaaacatgaagttaaaaactctagaaaagtaaaatgttcgctagaatggtataagctatttgagaatctcttattgttaaaatagttgtgattatgcaagaggatgtgggatgagaaatgctttatgctctgagaagaaaaattatgtgcttaaaacaaatacagacttaacctacagttctaaaagatatattgagaattatttaatcaaatctcaatgaaatgttttatcatcagcaagtagactcagaaaaaagaaaaaaaaatttatcatcagaaacaaggtactctgttttaacattttagaacatgtatgacaaaggaattatgtgcctgatgatgataatatatatctaaaatgtgcctcagtttcaaatacgccagcccgggttgttgtgacaaaatgtggaagcaggcacaaagatagtgagaaagatggaatctgtatttcttgtttctgaatgtatagctattaacttcttggatattgtatttcatatttaaaataatgtatttcttttggcatgtttattgttaaaaatttctgttccaattattttgatcagggtctctatcttaaaagtaaaaataactcagtgagggtttggctaagagccttcattaaacagagtttaaacacccagataaactctgctgtttaaccagagttaaacccaccagcactccaggaaaggcaatgcattggaataaacaagatgaagcagtgagacttgaacagctccatcttcactgctctatgttcaagatggccagttcttaaaaggaatataaagataacccataaatataactgttcatctatggacagtaatacaggagacgtgaaattttgttgactatgaaatagtggtcactgtttaaactctttgggtttccatttctttatctgtagaatgagagtgctggacttaatctctaatgtctattctttcttttttaaagctttaaatgtctatgaatatctaaagttctgggtattcttacagaaacatctgatcatacttctgagtaaaactttctactgagtctgatgttcagttgctcagtattatttcaataacatttgtagcaacactcactgcaggaacaagggacatattccaagaaaaaaaaggaaggaaggaaggaggggagaaaaggaaacattattataggtcatgagagttcagggaaaattaatcatgatctttattattttagttcaaagtgtcttcttggaacacttgtataaaattgtagcattattattctatattattgctctgttgtatttacatatgcatctgagaatttagctaatatgaactatgtagtttataacttaataattatttattatatatttgattttaaatgttcatgtttatggcttcttatttaagacctgatcatattaaatactacccgcccagta

SEQ ID NO: 3 - DNA, 2127 bp, synthetic construct
  source 1..2127: mol_type = other DNA; note = nucleotide sequence for SV40LT gene
atggataaagttttaaacagagaggaatctttgcagctaatggaccttctaggtcttgaaaggagtgcctgggggaatattcctctgatgagaaaggcatatttaaaaaaatgcaaggagtttcatcctgataaaggaggagatgaagaaaaaatgaagaaaatgaatactctgtacaagaaaatggaagatggagtaaaatatgctcatcaacctgactttggaggcttctgggatgcaactgagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatccattttctgttattgaggaaagtttgccaggtgggttaaaggagcatgattttaatccagaagaagcagaggaaactaaacaagtgtcctggaagcttgtaacagagtatgcaatggaaacaaaatgtgatgatgtgttgttattgcttgggatgtacttggaatttcagtacagttttgaaatgtgtttaaaatgtattaaaaaagaacagcccagccactataagtaccatgaaaagcattatgcaaatgctgctatatttgctgacagcaaaaaccaaaaaaccatatgccaacaggctgttgatactgttttagctaaaaagcgggttgatagcctacaattaactagagaacaaatgttaacaaacagatttaatgatcttttggataggatggatataatgtttggttctacaggctctgctgacatagaagaatggatggctggagttgcttggctacactgtttgttgcccaaaatggattcagtggtgtatgactttttaaaatgcatggtgtacaacattcctaaaaaaagatactggctgtttaaaggaccaattgatagtggtaaaactacattagcagctgctttgcttgaattatgtggggggaaagctttaaatgttaatttgcccttggacaggctgaactttgagctaggagtagctattgaccagtttttagtagtttttgaggatgtaaagggcactggaggggagtccagagatttgccttcaggtcagggaattaataacctggacaatttaagggattatttggatggcagtgttaaggtaaacttagaaaagaaacacctaaataaaagaactcaaatatttccccctggaatagtcaccatgaatgagtacagtgtgcctaaaacactgcaggccagatttgtaaaacaaatagattttaggcccaaagattatttaaagcattgcctggaacgcagtgagtttttgttagaaaagagaataattcaaagtggcattgctttgcttcttatgttaatttggtacagacctgtggctgagtttgctcaaagtattcagagcagaattgtggagtggaaagagagattggacaaagagtttagtttgtcagtgtatcaaaaaatgaagtttaatgtggctatgggaattggagttttagattggctaagaaacagtgatgatgatgatgaagacagccaggaaaatgctgataaaaatgaagatggtggggagaagaacatggaagactcagggcatgaaacaggcattgattcacagtcccaaggctcatttcaggcccctcagtcctcacagtctgttcatgatcataatcagccataccacatttgtagaggttttacttgctttaaaaaacctcccacacctccccctgaacctgaaacataa

SEQ ID NO: 4 - DNA, 5351 bp, synthetic construct
  source 1..5351: mol_type = other DNA; note = nucleotide sequence for hTERT gene
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggacaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggac

SEQ ID NO: 5 - DNA, 1942 bp, synthetic construct
  source 1..1942: mol_type = other DNA; note = Nucleotide sequence encoding CD19
aagctgggtgccccggagagtctgaccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggcagggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtgatcctcaggtggccagcctggatctcctcaagtccccaagattcacacctgactctgaaatctgaagacctcgagcagatgatgccaacctctggagcaatgttgcttaggatgtgtgcatgtgtgtaagtgtgtgtgtgtgtgtgtgtgtgtatacatgccagtgacacttccagtcccctttgtattccttaaataaactcaatgagctcttccaacctcccaggcagctcacagtg